(19) United States
(12) Patent Application Publication
(10) Pub. No.: US 2010/0278861 A1
(43) Pub. Date: Nov. 4, 2010

(54) METHODS AND COMPOSITIONS FOR PREDICTING EMERGENCE AND EXPANSION OF DRUG RESISTANT STRAINS OF INFLUENZA VIRUS

(76) Inventor: Henry L. NIMAN, Pittsburgh, PA (US)

Correspondence Address: LAHIVE & COCKFIELD, LLP;FLOOR 30, SUITE 3000, ONE POST OFFICE SQUARE, BOSTON, MA, 02109, US

(21) Appl. No.: 12/696,899

(22) Filed: Jan. 29, 2010

Related U.S. Application Data
(60) Provisional application No. 61/148,150, filed on Jan. 29, 2009.

(51) Int. Cl. A61K 39/145; C07H 21/02; C07K 14/11; A61P 31/16; C12Q 1/70

(52) U.S. Cl. 424/210.1; 536/23.72; 530/350; 424/209.1; 435/5

(57) ABSTRACT

The instant invention provides methods for determining, predicting and characterizing the genetic variability, emergence and expansion of viruses, in particular, influenza. Accordingly, the invention provides methods for identifying virulent pathogens, genetic mutations within pathogens that are relevant to animal health, and methods and compositions for prophylactic or therapeutic intervention against such pathogens.

### RELATED APPLICATIONS

This application claims priority to U.S. Patent Application No. 61/148,150, filed on Jan. 29, 2009. The entire contents of each of the foregoing application is expressly incorporated herein by reference.

The contents of all references, patents and published patent applications cited throughout this application are hereby incorporated herein by reference in their entirety.

### BACKGROUND

Despite remarkable achievements in the development of molecular genetics for understanding human and animal disease as well as determining the genetic nature of pathogens, rules for prediction or prognosis of future disease and pathogen virulence remain elusive. Typically, genetic alterations in cell genomes resulting in disease (or disease susceptibility) or genetic sequence of virulent pathogens is collected a posteriori, cataloged, and then used to make a diagnosis, and determine an appropriate therapeutic. Accordingly, responding therapeutically to the sequelae of genome instability in a subject suffering from a genetic disease (or disease susceptibility) and/or pathogen is typically reactive rather than proactive. This is especially true in treating, for example, human and animal response to pathogens such as viruses.

Development of viral vaccines presents unique challenges to modern medicine (see, for example, Ault, A. (2004) Science, 303:1280). Due to the constant evolution of many pathogens, and in particular, viruses, development of effective vaccines is often a difficult, and imperfect, process. Reliable prediction of the future molecular evolution of viral genomes would be expected to advance humankind's ability to combat such pathogens both prophylactically and therapeutically.

Viruses are the smallest of parasites, and are completely dependent upon the cells they infect for their reproduction. Of the viruses that infect humans, many infect their hosts without producing overt symptoms, while others (e.g., influenza A) produce a well-characterized set of symptoms. Importantly, although symptoms can vary with the virulence of the infecting strain, identical viral strains can have drastically different effects depending upon the health and immune response of the host.

A better understanding of the molecular events that lead to genome instability not only for understanding human and animal disease but also the evolution of pathogens such as viruses, is needed. Indeed, the ability to predict the molecular evolution of pathogenic genomes would be broadly expected to enhance the design of anti-pathogenic agents.

### SUMMARY OF THE INVENTION

Influenza has been viewed as a model for rapid genetic evolution and pandemic change, and has been the subject of intense research (Peiris, J. S. et al. Lancet 363, 617-619 (2004); Fouchier, R. et al. Nature 435, 419-420 (2005); Osterholm, M. T., N. Engl. J. Med. 352, 1839-1842 (2005); Monto, A. S., N. Engl. J. Med. 352, 32-325 (2005)). The conventional wisdom explains the evolution through selection of frequent copy errors generated by a polymerase complex that lacks a copy function (Webster, R. G. et al. Microbiol. Rev. 56, 152-179 (1992)). The errors are then selected for an evolutionary advantage, such as evasion of the immune response of the host, which allows the influenza to expand and fix the selected mutation. Similar selection of mutation generate variants has been used to describe drug resistance in viruses, prokaryotes, and eukaryotic cells.

However, close examination of emerging influenza genomes has determined that influenza employs recombination for rapid evolution via homologous recombination. This recombination is most common between closely related genomes because the increased regions of identity create additional opportunities. However, selection also plays a role, as indicated by large portions of influenza genes that have been faithfully replicated for over 25 years (Karasin, A. I. et al. J. Clin. Microbiol. 44, 1123-1126 (2006)). This unusually high level of fidelity also supports evolution via recombination, rather than selection of mutations.

In addition to use of frequent mutations for escape from immune recognition, similar mechanisms are thought to be involved in viral resistance. The identification of mutations involved in the development of resistance has also received considerable attention. Recently, the emergence of H5N1 with resistance to the antiviral drug, oseltamivir, was described in several patients infected with H5H1 (Le Q. M., et al. Nature 437:1108 (2005); de Jong, M. D., et al. N. Engl. J. Med. 353:2667-72 (2005). In each reported instance, H5H1 was isolated with the neuraminidase polymorphism, H274Y. This change was expected, because it is located in the active site of the enzyme, and oseltamivir binding requires a conformational change in the active site (Moscona, A., N. Engl. J. Med., 353:2633-2636 (2005)), and H274Y inhibits this change leading to an IC₅₀ increase of approximately 1000 fold. Thus, the H274Y was thought to be due to a copy error followed by selection. In one case, the H274Y change was accompanied by another change, N294S. This change was also expected because N294S is also located in the neuraminidase active site, and it also limits the conformational change, leading to an IC₅₀ increase of 10-15 fold.

Recently, the N294S change was found in two H5H1 infected patients (A/Egypt/14724-NAMRU3/2006 and A/Egypt/14725-NAMRU3/2006). Clinical samples were collected prior to oseltamivir treatment. Moreover, N294S has been detected in ducks (A/duck/Zhejiang/bj/2002 and A/Duck/Hong Kong/380.5/2001) that had not been treated with oseltamivir. Similarly, H274Y has also been found in H5H1 from a chicken (A/chicken/Hong Kong/3123.1/2002) and wild swan (A/swan/Astrakhan/Russia/Nov-2/2005) that had not been treated with oseltamivir.

These findings support the hypothesis that the drug resistance identified in the patients was due to changes that were present prior to treatment with oseltamivir. The polymorphisms were acquired via recombination, because they appeared on a genetic background that had region polymorphisms linked to H5H1 found in Egypt, the location of the recent oseltamivir resistant changes. Thus, like the seasonal changes found in influenza, the drug resistant polymorphisms are acquired by recombination and in cases where there are multiple versions of the recombinants, those that offer a selective advantage become dominant, as seen in the patient with H5H1 containing both H274Y and N294S.

The mechanism of recombination to create new recombinants with selectable polymorphisms can be used in testing for the identification of drug resistance and selection of drug development candidates. The screenings can be tested against parental strains that can form recombinants that allow for the escape from effects of the drug, and place these resistances changes of a favorable genetic background, as seen in the strains evolving in the patients in Egypt. These newly formed recombinants had the oseltamivir resistance, but on a regional genetic background.

The above example is illustrative of the use of recombination to create a new genetic entity with desirable properties of parental sequences. It has more predictive value than approaches that rely on mutational frequencies, because it relies on the existence and frequency of interactions coupled with selection pressures to determine the likelihood or frequency of the new entity which combines the desirable genetic aspects of the parental sequences.

This approach can be applied to all systems influenced by the appearance of genetic changes that have been previously thought to be due random mutations. Moreover, the frequency of sequences with desirable traits can be determined through exposure to the drug in question, and these frequencies can be used to predict future resistance.

Accordingly, the instant invention relates to a method of predicting emergence or expansion of a drug resistant viral strain sequence from sequences of a first parental viral strain and a second parental viral strain, comprising identifying a first parental viral strain sequence comprising one or more sequences correlated with a characteristic of the virus; identifying a second parental viral strain sequence lacking one or more of the one or more sequences of the first parental viral strain; and predicting drug resistant viral strain sequences capable of arising from a genetic transfer event comprising replacement of a second parental viral strain sequence with a first parental viral strain sequence. In one embodiment, the emergence of a drug resistant viral strain sequence is predicted. In one embodiment, the expansion of a drug resistant viral strain sequence is predicted.

In one embodiment, the viral strain sequence is resistant to a neuraminidase inhibitor, such as, for example, oseltamivir, zanamivir or peramivir. In certain embodiments, the viral strain sequence is resistant to an M2 inhibitor, such as, for example, amantadine or rimantadine.

In certain embodiments, the viral strains are influenza viruses. In one embodiment, the characteristic is genotypic, phenotypic, molecular, epidemiological, clinical, or pathological. In a related embodiment, the molecular characteristic is a nucleic acid alteration or amino acid alteration. In certain embodiments, the nucleic acid or amino acid alteration is in an influenza sequence selected from the group consisting of HA, NA, NP, PA, PB1, PB2, M1, M2, NS1, and NS2, or combinations thereof. In specific embodiments, the nucleic acid or amino acid alteration is in an influenza sequence selected from the group consisting of HA, NA, NP, PA, PB1, PB2, M1, M2, NS1, and NS2, or combinations thereof, as set forth in any of the Tables disclosed in International Patent Application No. PCT/US2006/026354, hereby incorporated herein by reference in its entirety. In one particular embodiment, the viral strain is H5H1. In a certain embodiment, the alteration causes at least about a 15-fold increase in drug resistance of the viral strain sequence compared to the with the wild-type viral strain sequence.

In one embodiment, the nucleic acid or amino acid alteration is in an influenza NA sequence, and in one particular embodiment, the alteration is an amino acid residue in an influenza NA sequence. In certain embodiments, the alteration is at residue 274 and is an alteration of histidine to tyrosine. In certain other embodiments, the alteration is at residue 294 and is an alteration of asparagine to serine. In certain embodiments, the alteration is at residue 31 of M2 and is an alteration of serine to asparagine. In certain other embodiments, the alteration is at residue 223 and is an alteration of valine to isoleucine. In another embodiment, the nucleic acid or amino acid alteration is in an influenza HA or NA sequence as set forth in any of the Tables disclosed in International Patent Application No. PCT/US2006/026354, hereby incorporated herein by reference in its entirety.

In one embodiment, the nucleic acid or amino acid alteration affects neuraminidase.

In an additional embodiment, the molecular characteristic is selected from the group consisting of viral infectivity, viral antigenicity, viral replication, and viral binding to a host cell receptor. In certain embodiments, the binding of the first parental viral strain to a cellular receptor is altered, as compared to the binding of the second parental viral strain to the cellular receptor. In specific embodiments, the binding is determined using a glycan chip assay. In another embodiment, the host cell receptor is an a 2-6-linked sialic acid glycoprotein.

In a further embodiment, the first parental viral strain sequence infects a host animal of a population of a first geographic range and the second parental viral strain sequence infects a host animal of a population of a second geographic range. In one embodiment, at least one of the first or second parental viral strain sequences is isolated from a host animal. In certain embodiments, the first and second geographic ranges do not overlap. In another embodiment, the host animals of the first and second parental viral strains are of different species. In one embodiment, at least one of the host animals of the first or second parental viral strains is a migratory bird. In a related embodiment, at least one of the host animals of the first or second parental viral strains is a migratory bird with a geographic range selected from the group consisting of North Africa, Europe, Asia, Middle East, Near East, North America, South America, and combinations thereof. In an additional embodiment, at least one of the host animals is avian. In certain embodiments, the animal is selected from the group consisting of a duck, chicken, turkey, ostrich, quail, swan, and goose. In additional embodiments, at least one of the host animals is selected from the group consisting of swine, chicken, duck, sheep, cattle, goat, and human. In one embodiment, at least one of the host animals is swine.

In certain embodiments, the first and second geographic ranges are projected to overlap within a time span selected from the group consisting of about a day, about a week, about 1 month, about 2 months, about 3 months, about 5 months, about 7 months, about 9 months, about 12 months, and ranges or intervals thereof. In one embodiment, the first and second parental viral strains are not predicted to have occupied the same geographic range. In an additional embodiment, the first and second geographic ranges are newly-overlapping. In further embodiments, the influenza is selected from the group consisting of influenza A, influenza B, and influenza C. In certain embodiments, the acceptor viral strain is selected from the group consisting of influenza A, influenza B and influenza C. In another embodiment, the genetic transfer event is a recombination-mediated genetic transfer event. In an additional embodiment, the genetic transfer event is occurs or is identified from cells cultured in vitro with one or more viral strains. In certain embodiments, the genetic transfer event involves a non-genomic DNA or RNA intermediate.

In another embodiment, the length of the first parental viral strain sequence is selected from the group consisting of about 5-10 nucleotides, about 10-20 nucleotides, about 10-20 nucleotides, about 20-50 nucleotides, about 50-100 nucleotides, about 100-1000 nucleotides, about 10-20 nucleotides, about 10-20 nucleotides, and ranges or intervals thereof. In certain embodiments, the first sequence and second sequence are at least 30% identical, at least 40% identical, at least 50% identical, at least 70% identical, at least 80% identical, at least 90% identical, at least 95%, at least 95%, at least 97%, at least 99% or ranges or intervals thereof. In an additional embodiment, the method further comprises producing a therapeutic compound or vaccine to at least one drug resistant viral strain. In another embodiment, the method further comprises administration of the therapeutic compound or vaccine to a subject.

In an additional embodiment, the invention relates to a sequence identified according to any of the methods of the invention that is suitable for use in the development of a prognostic compound, diagnostic compound, therapeutic compound, or vaccine. In certain embodiments, the sequence comprises one or more sequences as set forth in any of the Tables disclosed in International Patent Application No. PCT/US2006/026354, hereby incorporated herein by reference in its entirety.

In another embodiment, the invention relates to a composition comprising a nucleic acid or polypeptide sequence identified according to the methods of the invention.

A further aspect of the invention relates to a composition comprising an influenza nucleic acid or polypeptide sequence having an alteration as set forth in any of the Tables disclosed in International Patent Application No. PCT/US2006/026354, hereby incorporated herein by reference in its entirety. In one embodiment, the nucleic acid or polypeptide sequence is an altered influenza NA sequence. In certain embodiments, the alteration is at residue 274 and is an alteration of histidine to tyrosine. In certain other embodiments, the alteration is at residue 294 and is an alteration of asparagine to serine. In certain other embodiments, the alteration is at residue 223 and is an alteration of valine to isoleucine. In certain embodiments, alteration is at residue 31 and is an alteration of methionine to isoleucine. In an additional embodiment, the nucleic acid or polypeptide sequence comprises an alteration in an influenza NA sequence.

Another embodiment of the invention relates to a vaccine composition comprising an altered influenza nucleic acid or polypeptide sequence according to any of the methods or compositions of the invention. An additional embodiment relates to a method of immunizing an animal or human subject against influenza comprising administering such a vaccine composition to the subject.

A further aspect of the invention relates to a kit for predicting or identifying the occurrence of an influenza virus strain comprising an influenza sequence or influenza composition as set forth in any of the Tables disclosed in International Patent Application No. PCT/US2006/026354, hereby incorporated herein by reference in its entirety.

In another embodiment, the invention provides for a comparison of parental viral strains with their mutant drug resistant viral strains which can be used to define and elucidate selective pressures on rapid evolution. The identification of recombinants can be used to identify genetic instability, which is currently evident in many viruses throughout the world, for example, influenza A and influenza B. The parental viruses can also be used to create recombinants prior to detection in field isolates and such recombinants can be used to make protective vaccines against future recombinants, which cause significant disruptions in animal husbandry and human health.

In still another embodiment, the invention provides rules that can be applied, e.g., to predict the genetic composition and, optionally, associated phenotypic traits (e.g., drug resistance) of viruses or bacteriae that arise from the mixing within a single host organism of distinct “parental” viruses or bacteriae (e.g., ebola, flu and/or HIV; foot and mouth and Newcastle disease; SARS, HIV and/or astroviruses; HIV and coronavirus; distinct drug-resistant bacterial strains, etc.).

In yet another embodiment, the invention provides methods of generating libraries of diverse viral sequences to be used, for example, in the manufacture of viral vaccines, or for testing of antiviral compounds. The invention further provides methods of identifying parental viral strains.

The instant invention also provides methods for monitoring the efficacy of viral vaccines and for monitoring the diversity of a viral population.

Accordingly, the invention has several advantages, which include, but are not limited to, the following:
providing methods for determining gene sequences in a human or animal suitable for modulating thereby preventing or treating a disease or disorder;
methods and compositions relating to the development of therapeutics against pathogen targets, for example, viral pathogens; and
methods and compositions relating to the development of therapeutics against pathogens, for example, viral pathogens, having acquired or susceptible for acquiring a genetic transfer event from another pathogen, for example, viral pathogen, and/or host cell.

In one aspect, the invention provides methods of predicting emergence or expansion of a drug resistant viral strain sequence from sequences of a first parental viral strain and a second parental viral strain, comprising identifying a first parental viral strain sequence comprising one or more sequences correlated with a characteristic of the virus; identifying a second parental viral strain sequence lacking one or more of the one or more sequences of the first parental viral strain; and predicting drug resistant viral strain sequences capable of arising from a genetic transfer event comprising replacement of a second parental viral strain sequence with a first parental viral strain sequence, such that emergence or expansion of a drug resistant viral strain sequence having a characteristic of the parental viral strain is predicted.

In one embodiment, the viral strains are influenza viruses. In one embodiment, the influenza is selected from the group consisting of influenza A, influenza B, and influenza C. In one embodiment, the influenza virus is H5H1.

In one embodiment, the viral strain sequence is resistant to a neuraminidase inhibitor. In another embodiment, the neuraminidase inhibitor is oseltamivir, zanamivir or peramivir.

In one embodiment, the viral strain sequence is resistant to an M2 inhibitor. In another embodiment, the M2 inhibitor is amantadine or rimantadine.

In one embodiment, the alteration affects neuraminidase. In another embodiment, the alteration causes at least about a 15-fold increase in drug resistance of the viral strain sequence compared to the with the wild-type viral strain sequence.

In another embodiment, the characteristic is genotypic, phenotypic, molecular, epidemiological, clinical, or pathological. In another embodiment, the molecular characteristic is a nucleic acid alteration or amino acid alteration.

In one embodiment, the nucleic acid or amino acid alteration is in an influenza sequence selected from the group consisting of HA, NP, NA, PA, PB1, PB2, M1, M2, NS1, and NS2, or combinations thereof. In another embodiment, the nucleic acid or amino acid alteration is in an influenza sequence selected from the group consisting of HA, NP, NA, PA, PB1, PB2, M1, M2, NS1, and NS2, or combinations thereof, as set forth in any of the Tables herein.

In one embodiment, the nucleic acid or amino acid alteration is in an influenza NA sequence. In another embodiment, the alteration is an amino acid residue in an influenza NA sequence. In another embodiment, the alteration is at amino acid residue 274. In another embodiment, the alteration of amino acid 274 is histidine to tyrosine. In another embodiment, the alteration is at amino acid residue 294. In another embodiment, the alteration of amino acid 294 is asparagine to serine.

In one embodiment, the viral strain further comprises additional alterations at amino acid residue 344 and amino acid residue 354. In one embodiment, the additional alteration and amino acid residue 344 is Aspartic Acid to Asparagine, and wherein the additional alteration in amino acid residue 354 is Aspartic Acid to Glycine.

In one embodiment, the alteration is at amino acid residue 223 of HA. In another embodiment, the alteration of amino acid 223 of HA is valine to isoleucine.

In one embodiment, the amino acid alteration is an alteration in amino acid residue 196 of HA. In another embodiment, the alteration in amino acid residue 196 of HA is histidine to arginine or glutamine to arginine.

In one embodiment, the nucleic acid or amino acid alteration is in an influenza M2 sequence. In another embodiment, the alteration is an amino acid residue in an influenza M2 sequence. In another embodiment, the alteration is at amino acid residue 31. In another embodiment, the alteration of amino acid 31 is Serine to Asparagine.

In another embodiment, the viral strain further comprises an additional alteration at amino acid residue in an influenza HA sequence selected from the group consisting of: amino acid residue 192, amino acid residue 193, and amino acid residue 197. In another embodiment, the viral strain further comprises additional alterations in an influenza HA sequence at amino acid residues 192, 193 and 197. In one embodiment, the alteration of amino acid residue 192 is Arginine to Methionine, wherein the alteration of amino acid residue 193 is Alanine to Threonine, and wherein the alteration of amino acid residue 197 is Threonine to Lysine.

In another embodiment, the viral strain further comprises an additional alteration at an amino acid residue in an influenza HA sequence. In one embodiment, the additional alteration is at amino acid residue 225 or 193. In one embodiment, the additional alterations are at amino acid residue 225 and amino acid residue 193. In one embodiment, the additional alteration of amino acid residue 225 is Aspartic Acid to Asparagine, and wherein the additional alteration of amino acid residue 193 is Serine to Phenylalanine.

In another embodiment, the viral strain further comprises additional alterations in an influenza NA sequence at amino acid residue 344 and amino acid residue 354. In one embodiment, the additional alteration at amino acid residue 344 is Aspartic Acid to Asparagine, and wherein the additional alteration at amino acid residue 354 is Aspartic Acid to Glycine.

In one embodiment, the nucleic acid or amino acid alteration is in an influenza HA sequence. In one embodiment, the alteration is an amino acid residue in an influenza HA sequence. In another embodiment, the alteration is at amino acid residue 187. In another embodiment, the alteration is at amino acid residue 189. In another embodiment, the alteration is at amino acid residue 193. In another embodiment, the alteration of amino acid 187 is Asparagine to Aspartic Acid. In another embodiment, the alteration of amino acid 189 is Glycine to Asparagine. In another embodiment, the alteration of amino acid 193 is Alanine to Threonine.

In another embodiment, the viral strain further comprises an additional alteration at amino acid residue 190. In another embodiment, the viral strain further comprises an additional alteration at amino acid residue 189. In another embodiment, the viral strain further comprises an additional alteration at amino acid residue 187. In one embodiment, the alteration of amino acid residue 190 is Aspartic Acid to Asparagine. In one embodiment, the alteration of amino acid residue 189 is Glycine to Valine. In one embodiment, the alteration of amino acid residue 187 is Asparagine to Aspartic Acid.

In another embodiment, the viral strain further comprises additional alterations at amino acid residue 190 and amino acid residue 189. In one embodiment, the alteration of amino acid residue 190 is Aspartic Acid to Asparagine, and wherein the alteration of amino acid residue 189 is Glycine to Valine.

In one embodiment, the alteration is at amino acid residue 223. In another embodiment, the alteration of amino acid 223 is valine to isoleucine.

In one embodiment, the alteration is at amino acid residue 31. In another embodiment, the alteration of amino acid 31 is methionine to isoleucine.

In one embodiment, the molecular characteristic is selected from the group consisting of viral infectivity, viral antigenicity, viral replication, and viral binding to a host cell receptor.

In one embodiment, the binding of the first parental viral strain to a cellular receptor is altered, as compared to the binding of the second parental viral strain to the cellular receptor. In another embodiment, the binding is determined using a glycan chip assay. In another embodiment, the host cell receptor is an α2-6-linked sialic acid glycoprotein.

In one embodiment, at least one of the first or second parental viral strain sequences is isolated from a host animal. In one embodiment, the host animals of the first and second parental viral strains are of different species. In another embodiment, at least one of the host animals of the first or second parental viral strains is a migratory bird. In one embodiment, at least one of the host animals of the first or second parental viral strains is a migratory bird with a geographic range selected from the group consisting of North Africa, Europe, Asia, Middle East, Near East, North America, South America, and combinations thereof. In one embodiment, at least one of the host animals is avian. In another embodiment, the animal is selected from the group consisting of a duck, chicken, turkey, ostrich, quail, swan, and goose. In another embodiment, at least one of the host animals is selected from the group consisting of swine, chicken, duck, sheep, cattle, goat, and human. In another embodiment, at least one of the host animals is swine.

In one embodiment, the first parental viral strain sequence infects a host animal of a population of a first geographic range and the second parental viral strain sequence infects a host animal of a population of a second geographic range. In one embodiment, the first and second geographic ranges do not overlap. In another embodiment, the first and second geographic ranges are projected to overlap within a time span selected from the group consisting of about a day, about a week, about 1 month, about 2 months, about 3 months, about 5 months, about 7 months, about 9 months, about 12 months, and ranges or intervals thereof. In another embodiment, the first and second parental viral strains are not predicted to have occupied the same geographic range. In another embodiment, the first and second geographic ranges are newly-overlapping.

In one embodiment, the genetic transfer event is a recombination-mediated genetic transfer event. In another embodiment, the genetic transfer event occurs or is identified from cells cultured in vitro with one or more viral strains. In one embodiment, the genetic transfer event involves a non-genomic DNA or RNA intermediate.

In one embodiment, the length of the first parental viral strain sequence is selected from the group consisting of about 5-10 nucleotides, about 10-20 nucleotides, about 10-20 nucleotides, about 20-50 nucleotides, about 50-100 nucleotides, about 100-1000 nucleotides, about 10-20 nucleotides, about 10-20 nucleotides, and ranges or intervals thereof.

In one embodiment, the first sequence and second sequence are at least 30% identical, at least 40% identical, at least 50% identical, at least 70% identical, at least 80% identical, at least 90% identical, at least 95%, at least 95%, at least 97%, at least 99% or ranges or intervals thereof.

In one embodiment, the method further comprises producing a therapeutic compound or vaccine to at least one drug resistant viral strain. In one embodiment, the method further comprises administration of the therapeutic compound or vaccine to a subject.

In another aspect, the invention provides a sequence identified according to any of the foregoing methods suitable for use in the development of a prognostic compound, diagnostic compound, therapeutic compound, or vaccine.

In another aspect, the invention provides a composition comprising a nucleic acid or polypeptide sequence identified according to the methods of the invention. In one embodiment, the nucleic acid or polypeptide sequence is an altered influenza HA sequence. In another embodiment, the alteration is an amino acid residue in an influenza HA sequence. In another embodiment, the alteration is at amino acid residue 193. In another embodiment, the alteration of amino acid residue 193 is Alanine to Threonine.

In one aspect, the invention provides a composition comprising a nucleic acid or polypeptide sequence, wherein the nucleic acid or polypeptide sequence is selected from the group consisting of: an altered influenza NA sequence, an altered influenza M2 sequence, and an altered influenza HA sequence. In one embodiment, the alteration is an amino acid residue in an influenza NA sequence. In one embodiment, the alteration is at amino acid residue 274. In another embodiment, the alteration of amino acid 274 is histidine to tyrosine. In another embodiment, the alteration is at amino acid residue 294. In another embodiment, the alteration of amino acid 294 is asparagine to serine.

In one embodiment, the alteration is an amino acid residue in an influenza HA sequence. In another embodiment, the alteration is at amino acid residue 193. In another embodiment, the alteration of amino acid 193 is alanine to threonine. In another embodiment, the alteration is an amino acid residue in an influenza M2 sequence. In one embodiment, the alteration is at amino acid residue 31. In another embodiment, the alteration of amino acid residue 31 is serine to asparagine.

In another aspect, the invention provides a vaccine composition, comprising an altered influenza nucleic acid or polypeptide sequence according to any of the above claims. In another aspect, the invention provides a method of manufacturing such a vaccine composition. In another aspect, the invention provides a kit comprising the vaccine composition and instructions for use.

In another aspect, the invention provides a method of immunizing an animal or human subject against influenza comprising administering to the subject any of the compositions of the invention described herein.

In one aspect, the invention provides a kit for predicting or identifying the occurrence of an influenza virus strain comprising an influenza sequence or influenza composition and instructions for their use.

In one embodiment, one or more steps of the invention is computer-assisted. In another embodiment, the invention provides a medium suitable for use in an electronic device having instructions for carrying out one or more steps of the methods of the invention as described herein. In another embodiment, the invention provides a device for carrying out one or more steps of the method of the invention.

### BRIEF DESCRIPTION OF THE DRAWINGS

FIG. 1 depicts an NA Phylogram (positions 601-1095) of H1N1 Isolates. Trees were generated using neighbor joining and 100 bootstrap repetitions. Isolates with H274Y are marked with (*). Isolates with known S31N on M2 are marked with (@). A. Expansion of clade 1, 2B, 2C. B. Non-synonymous polymorphisms are in bold, italics. Synonymous polymorphism are underlined. D344N encoded by G1030A. D354G encoded by A1061G.

FIG. 2 depicts an HA Phylogram (positions 69-632) of H1N1 and H1N2 Isolates. Trees were generated using neighbor joining and 100 bootstrap repetitions. Isolates with H274Y are marked with (*). Isolates with known S31N on M2 are marked with (@). A. Expansion of clade 1, 2B, 2C. B. N187S encoded by A599G. G189N encoded by G604A and G605A. R192M encoded by G614A. A193T encoded by G616A. T197K encoded by C629A.

Other features and advantages of the invention will be apparent from the following detailed description and claims

### DETAILED DESCRIPTION

In order to provide a clear understanding of the specification and claims, the following definitions are conveniently provided below.

### Definitions

The term, “parental viral strains” is intended to mean the two, or more, viral strains in a population that supply the genetic material to the drug resistant viral strains in the population through a copy choice recombination mechanism. The parental viral strains are two or more strains of virus that are present in a recently (e.g., within one, two, three, six, twelve, or more months) isolated population of viruses. In one aspect the parental viral strains are the most prevalent sequences in a population. In another aspect, the parental viral strains are the most diverse sequences in a population.

The term “drug resistant viral strain” refers to those viral strains that have mutated, via one or more genetic mutations, which renders a drug administered to a subject to counteract the viral strain, ineffective. The drug resistant viral strain may be resistant to one or more drugs or other pharmaceutical agents. In one embodiment, the viral strain is drug resistant to a neuraminidase inhibitor (e.g., oseltamivir, zanamivir or peramivir). In another embodiment, the viral strain is drug resistant to an M2 inhibitor (e.g., amantadine or rimantadine).

The term “emergence” refers to the increasing appearance of new viral strains. The term “expansion” refers to the increasing in volume or quantity of a new viral strain.

The term “copy choice recombination” as used herein is intended to mean the mechanism of viral or bacterial recombination in which a drug resistant virus is made in a cell or organism that has been infected by two or more parent viral strains and the genetic material of the drug resistant virus is a mix of the genetic material of the parent strains. Without being bound by mechanism, copy choice mechanism results from the DNA or RNA replication machinery starting on DNA or RNA from one parent and switching to the DNA or RNA from a second parental strain during duplication of a piece of DNA or RNA. This process can happen one or more times thereby resulting in progeny virus or bacteria that has a DNA or RNA sequence that is a mix of the two parental strains.

Sequences produced by copy-choice recombination, e.g., drug resistant sequences, can contain any number of nucleotide changes, including one or more nucleotide changes as compared with parental sequences, e.g., 2-5, 5-10, 10-20, 20-50, 50-100, 100-500, 500 or greater changes, typically by recombination, e.g. copy-choice recombination, occurring within a given length of nucleic acid, between two or more strands of nucleic acid, e.g., within two nucleotides or more, e.g., 3-5, 5-10, 10-100, 100-1 kb, 1 kb-10 kb, 10 kb or more, or any range or interval thereof.

The term “transition/transversion ratio” as used herein is intended to denote a ratio between the number of times a given sequence has a transition, e.g., the substitution of a purine for a purine, or a pyrimidine for a pyrimidine, versus the number of times the sequence has a transversion, e.g., a purine for a pyrimidine or a pyrimidine for a purine. One would expect the ratio to be 0.5 if it were a random process. However, looking at multiple data sets, it has been determined that the ration is often 2 or higher, indicative that the process is not random and that transitions are favored over transversions (see the Exemplification).

The term “genetic transfer event” refers to an exchange of sequence information between two or more gene loci. Such sequence transfer may be inter- or intragenic, in cis or in trans, and/or between one or more species of pathogens (e.g., viral pathogens) and/or cells (e.g., host cells or organisms).

The present invention, at least in part, is based on the surprising observation that recombination, rather than de novo mutation, is a driving force of viral evolution. In particular, the present invention, at least in part, is based on the observation that pathogens can exchange nucleic acid sequence intergenically or intragenically between one or more pathogens and/or cells, e.g., host cells, with which the pathogens can reside (or infect and/or co-infect). In one observation, drug resistant strains of influenza are effectively derived as haplotypes from divergent, “parental” strains of influenza A and/or influenza B, revealing that dual infections of a single cell or organism with two or more distinct strains of virus (or distinct types of virus, e.g., influenza and HIV, or distinct strains of bacteria) can accelerate viral evolution. The present invention therefore provides rules for predicting the outcome of such real-world or controlled mixing experiments. In certain aspects of the invention, these rules can be applied to predict drug resistant influenza A and/or influenza B strains that represent optimal vaccine targets, based upon knowledge (optionally real-time knowledge) of the genetic makeup of the prevalent influenza A and/or influenza B strains in a population. In other observations, other viral pathogens are identified as having acquired a genetic transfer event.

In one aspect, the rules of the invention may be applied to enable prediction of the genomic composition and/or phenotypic traits e.g., drug resistance of viral strains derived from at least two parental strains of virus. Such drug resistant viruses can then be used, e.g., in subsequent drug screening and/or vaccine development steps.

In one aspect, the instant invention provides a method for identifying parental viral strains in a population of viruses, wherein the population comprises parental viral strains and drug resistant viral strains, comprising the steps of: obtaining the nucleic acid or polypeptide sequence of one or more viral genes from a number of isolated viral strains from the population, the number sufficient to allow for identification of the viral strains most prevalent in the population, the viral strains having the greatest sequence divergence in the population, or both; identifying the viral strains most prevalent in the population, or viral strains with the greatest sequence divergence in the population, or both; wherein the most prevalent viral sequences, or the viral sequences with the greatest divergence are the parental viral strains.

In one embodiment, the parental viral strains are the two most prevalent sequences in the population. In another embodiment, the parental strains are the two strains with greatest sequence divergence.

In one embodiment, the viruses used in the methods of the invention are from a period of time sufficient to allow for the determination of the parental and drug resistant viral strains. For example, the period of time in which isolated viruses can be used in the methods of the invention can be 1 month, 2 months, 3 months, 4 months, 6 months, 1 year, 2 years, 3 years, 4 years, 5 years, or more. In one exemplary embodiment, the viruses used in the methods of the invention are from one outbreak season, e.g., one influenza season.

In another embodiment, the methods of the invention use viruses from a defined geographic area, e.g., one in which infected hosts have reasonable chance of interacting. For example, defined geographic areas are southeast Asia, or the continental United States.

In a related embodiment, the most prevalent viral sequences, or the viral sequences with the greatest sequence divergence are determined by aligning multiple nucleic acid or polypeptide sequences. In another related embodiment, the drug resistant viral strains are formed by recombination according to a copy-choice mechanism. In another embodiment, the viral sequence has acquired a genetic transfer event from another virus (e.g., strain or species) and/or host cell within which it can reside or infect.

Sequence alignments can be done using, for example, a mathematical algorithm. In one embodiment, the percent identity between two amino acid sequences is determined using the Needleman and Wunsch (J. Mol. Biol. 48:444-453 (1970)) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

Further, algorithms based upon sequence alignment programs can be developed that automatically compare viral sequences deposited in a database to determine the sequence identity in a population. Bioinformatic approaches can be used to monitor the amount of sequence diversity as a function of time, and location, thereby alerting medical professionals as to when their intervention, i.e., immunization, efforts should be increased. A Bioinformatics approach would be particularly useful for viral populations where there are large databases that would be difficult to align and/or sort, e.g., by date or location, manually, e.g., HIV or influenza A and/or influenza B. Bioinformatics can be used to determine the parental viral strains in a population of viruses and/or determine the mutant viral progeny viruses in a population of viruses by sorting the nucleic acid or polypeptide sequences by, for example, the number and/or location of non-identical nucleotides or amino acids, respectively.

In another aspect, bioinformatics can be used to evaluate databases of viral sequences to identify historically significant sequence variations in a viral gene sequence. The emergence of a previously identified sequence polymorphism is indicative of copy-choice recombination. Without being bound by mechanism, the emergence of a sequence polymorphism in a population of viruses that has not been observed for some time is a sign that there has been copy-choice recombination between two viruses. This approach will allow one of skill in the art to identify, in silico, drug resistant viral strains that may be problematic, e.g., have high infectivity. Further, analysis of viral sequences in a database for the presence of a known sequence polymorphism that is normally not found in a given geographic area can indicate that copy-choice recombination has occurred.

In one aspect, the methods of the invention may use a computer based program to identify multiple cross-over points in drug resistant viral strains. Due to the high number of cross-over points in some genes formed by copy choice recombination (often 10-100 cross-over points per gene) computer algorithms will be useful tools to determine the precise location of cross-over points. These computer algorithms can compare a large database of viral sequences to determine the location of cross-overs in a parental viral strain that gave rise to drug resistant viral strains. The precise mapping of these locations in combination with analysis of the various polymorphisms will allow one of skill in the art to classify viruses based on genotype rather than the serotype classification currently used.

### Computer Prediction Methods

The identification of influenza drug resistant strains of the present invention can also be conducted with the benefit of structural or modeling information concerning the sequences to be generated, such that the potential for generating drug resistant strains of importance for diagnostics and/or vaccine development is increased. The structural or modeling information can also be used to guide the selection of predetermined sequences to introduce into defined regions. Still further, actual results obtained with the present selection methods of the invention can guide the selection (or exclusion) of subsequent drug resistant to be identified, made and/or screened in an iterative manner. Accordingly, structural or modeling information can be used to generate initial subsets of progeny sequences for use in the invention as parental strains for future generations, thereby further increasing the efficiency of predicting progeny sequences.

In a particular embodiment, in silico modeling is used to eliminate the production of any sequence predicted to have poor or undesired structure and/or function. In this way, the number of drug resistant sequences identified and/or produced can be reduced, thereby increasing signal-to-noise in the drug resistant sequence output of the invention, optionally used in subsequent iterations of the methods of the invention. In another particular embodiment, the in silico modeling is continually updated with additional modeling information, from any relevant source, e.g., from gene databases (e.g., NCBI, Genbank, influenza sequence databases, etc.) and protein sequence and three-dimensional databases and/or results from previously tested sequences, so that the in silico database becomes more precise in its predictive ability. Accordingly, the methods of the invention may be run as, e.g., a macro capable of leveraging the sequence content of art-recognized sequence databases containing influenza sequence. Such a macro and/or computer-assisted program may be iteratively updated as additional sequences are deposited in sequence databases. In fact, as influenza databases continue to expand in content, the value of information produced via practice of the methods of the present invention is anticipated to rise.

In a preferred embodiment, one or more of the above steps are computer-assisted. The method is also amenable to be carried out, in part or in whole, by a device, e.g., a computer driven device. Accordingly, instructions for carrying out the method, in part or in whole, can be conferred to a medium suitable for use in an electronic device for carrying out the instructions. In sum, the methods of the invention are amendable to a high throughput approach comprising software (e.g., computer-readable instructions) and hardware (e.g., computers, robotics, and chips).

In one embodiment, drug resistant viral strains can be produced by a copy-choice recombination mechanism in combination with reassortment. In another embodiment, the drug resistant progeny viruses are produced by copy-choice recombination in the absence of reassortment.

Further, in vitro or in vivo techniques can be used to selectively recombine individual genes from different viruses in the population to produce drug resistant viruses. For example, a number of genes from a population of viruses can be analyzed using, for example, sequence alignments. One of skill in the art can isolate genes with desired sequences from the population and use those genes to infect a host cell, egg, or animal to produce a desired set of recombinants. In this situation the genes used to infect the host can come from multiple different viruses (e.g., 3, 4, 5, 6, 7, 8, 9, 10, 11, 12, 13, 14, 15 or more different viruses).

In one aspect of the invention, the methods of the invention can be used with any viruses that infect a subject. The term “subject” is intended to include organisms which are capable of having a viral infection. Examples of subjects include mammals, e.g., humans, dogs, cows, horses, pigs, sheep, goats, cats, mice, rabbits, rats, and transgenic non-human animals, or birds, e.g., ducks, chicken, geese, and swans. In certain embodiments, the subject is a human. Similarly, the term “host” is intended to include organisms, e.g., mammals, e.g., humans, dogs, cows, horses, pigs, sheep, goats, cats, mice, rabbits, rats, or birds, e.g., ducks, chicken, geese, and swans, and transgenic non-human animals, that harbor a viral strain, nucleotide sequences that recombine via copy-choice recombination, etc.

In one embodiment, the viruses are RNA viruses. In one embodiment, the RNA viruses are single-stranded RNA viruses. In one embodiment, the single-stranded RNA viruses are positive-sense RNA viruses. In another embodiment, the single-stranded RNA viruses a re negative-sense RNA viruses. In a related embodiment, the RNA viruses are double-stranded RNA viruses. In one related embodiment, the double-stranded RNA viruses are positive-strand RNA viruses. In another embodiment, the double-stranded RNA viruses are negative-strand RNA viruses.

In another embodiment, the viruses are DNA viruses. In one embodiment, the DNA viruses are single-stranded DNA viruses. In another embodiment, the DNA viruses are double-stranded DNA viruses.

In one embodiment, the viruses are influenza A and/or influenza B viruses. In another embodiment, the viruses are coronavirus viruses, e.g., SARS CoV.

In one embodiment, the protein or nucleic acid sequences are from influenza A and/or influenza B viruses.

In one embodiment, the virus is H5H1. In another embodiment, the virus is H1N1. In yet another embodiment, the virus is H3N2. In another embodiment, the virus is H1N2. In certain embodiments, the virus is drug resistant H5H1. In other embodiments, the virus is drug resistant H1N1. In other embodiments, the virus is drug resistant H3N2. In other embodiments, the virus is drug resistant H1N2.

In one embodiment, the nucleic acid or amino acid alteration is in an influenza NA sequence, and in one particular embodiment, the alteration is an amino acid residue in an influenza NA sequence. In certain embodiments, the alteration is at residue 274 and is an alteration of histidine to tyrosine. In certain other embodiments, the alteration is at residue 294 and is an alteration of asparagine to serine. In certain other embodiments, the alteration is at residue 223 and is an alteration of valine to isoleucine. In certain embodiments, alteration is at residue 31 and is an alteration of methionine to isoleucine. In another embodiment, the nucleic acid or amino acid alteration is in an influenza NA or HA sequence as set forth in any of the Tables disclosed in International Patent Application No. PCT/US2006/026354, hereby incorporated herein by reference in its entirety.

In one embodiment, the nucleic acid or amino acid alteration affects neuraminidase.

In one embodiment, the influenza A and/or influenza B nucleic acid or polypeptide sequences are selected from the group consisting of: HA, NA, NP, PA, PB1, PB2, MP, and NS, or combinations thereof.

In one embodiment, the nucleic acid or polypeptide sequences are obtained by sequencing the isolated viral strains. In another embodiment, the sequences are obtained by sequencing nucleic acid molecules isolated from a subject (e.g., a human or animal) or a tissue sample. In another embodiment, the nucleic acid or polypeptide sequences are obtained from a publicly available database. In certain embodiments, the sufficient number is 5, 10, 20, 30, 40, 50 or more viral sequences.

In one embodiment, the one or more viral genes is at least two, three, four or five or more genes.

In another aspect, the invention provides a method of producing a viral vaccine, comprising: infecting a host animal, host animal cell, cell line, egg cell, bacterial cell, or cell extract which supports viral replication with the parental viral strains identified according to the methods described above; and isolating drug resistant viral strains from the host animal cell line, egg cell, bacterial cell, or cell extract which supports viral replication.

Viral vaccines of the present invention can be, for example, live vaccines, killed vaccines, attenuated vaccines or subunit vaccines (see, for example, Fields Virology, (1996) Third Edition, Lippencott-Raven Publishers, Philadelphia, pp. 467-469.) Further examples of vaccine production are, for example, Meadors et al. (1986) Vaccine: 179-184, Poland et al. (1990) J. Infect. Disease 878-882, Fenner et al. The Biology of Animal Viruses; New York, Academic Press, 1974:543-586, Saban et al. (1973) J. Biol. Stand. 115-118, and Lowrie et al., DNA Vaccines: Methods and Protocols, Humana Press, N.J., 1999.

An attenuated whole organism vaccine uses a non-pathogenic form of the desired virus. Non-pathogenicity may be induced by growing the virus in abnormal conditions. Those mutants that are selected by the abnormal medium are usually limited in their ability to grow in the host and be pathogenic. The advantage of the attenuated vaccine is that the attenuated pathogen simulates an infection without conferring the disease. Since the virus is still living, it provides continual antigenic stimulation giving sufficient time for memory cell production. Also, in the case of viruses where cell-mediated immunity is usually desired, attenuated pathogens are capable of replicating within host cells. Genetic engineering techniques are being used to bypass these disadvantages by removing one or more of the genes that cause virulence.

An inactivated whole organism vaccine uses viruses which are killed and are no longer capable of replicating within the host. The viruses are inactivated by heat or chemical means while assuring that the surface antigens are intact. Inactivated vaccines are generally safe, but are not entirely risk free. Multiple boosters are usually necessary in order to generate continual antigen exposure, as the dead organism is incapable of sustaining itself in the host, and is quickly cleared by the immune system.

One or more polypeptides, or fragments thereof, that are presented by a virus can be formulated into a vaccine that elicits an immune response in a host. These so called “subunit” vaccines often alleviate the safety concerns associates with whole virus vaccines.

In a related embodiment, the method further comprises attenuating drug resistant viral strains to make an attenuated viral vaccine. In another embodiment, the method further comprises killing the drug resistant viral strains to make a killed viral vaccine. In another embodiment, the method further comprises isolating viral antigens, or portions thereof, from the drug resistant viral strains to make a subunit viral vaccine.

In another embodiment, the invention provides a method of immunizing a subject against a virus comprising: administering to the subject the attenuated virus vaccine in an amount sufficient to immunize the subject. In one embodiment, the subject is a mammal, e.g., a human, in another embodiment the subject is a bird. In one embodiment, the method of immunizing a subject (e.g., a human or animal) against a virus comprising: administering to the subject a killed, or attenuated, virus vaccine in an amount sufficient to immunize the subject. In another embodiment, the invention provides a method of immunizing a subject against a virus comprising administering to the subject the subunit virus vaccine in an amount sufficient to immunize the subject. In one embodiment, the parental strains are influenza A and/or influenza B viral strains. In another embodiment, the parental strains are coronavirus viral strains.

In one aspect, the invention provides a method of immunizing a subject (e.g., a human or animal) against a virus comprising: administering to the subject a first virus representing the first parental viral strain and a second virus representing a second parental viral strain, the first and second parental viral strains identified according to the methods described herein, in an amount sufficient to immunize the subject.

In one embodiment, the parental viral strains are attenuated prior to administering to the subject. In another embodiment, the parental viral strains are killed prior to administering to the subject. In another embodiment, the method comprises isolating viral antigens, or portions thereof, from the parental viral strains to make a subunit viral vaccine prior to administering to the subject.

In one embodiment, the parental viral strains are influenza A and/or influenza B viral strains. In another embodiment, the parental viral strains are coronavirus viral strains.

In another aspect, the invention provides a viral vaccine composition comprising the parental viral strains identified according to the methods described herein, or antigens, or portions of antigens, therefrom.

In one embodiment, the viral vaccine further comprises drug resistant viral strains derived from the parental viral strains, or antigens, or portions of antigens, therefrom. In another embodiment, the vaccine comprises two viral strains, or antigens, or portions of antigens from two viral strains.

In another embodiment, the vaccine composition, comprising drug resistant viral strains, or antigens or portions of antigens therefrom, is made by recombination according to a copy-choice mechanism of two viral strains whose genomes are made up of non-identical nucleic acid sequences. In one embodiment, the two viral strains are parental viral strains identified according to the methods described herein.

In one embodiment, the drug resistant viral strains are produced by recombination according to a copy-choice mechanism in a host animal. In another embodiment, the drug resistant viral strains are produced by recombination according to a copy-choice mechanism in cell culture.

In one embodiment, subjects who should be given a viral vaccine can be determined based on the genotype of the current viral strains in a population. Similarly, the type of vaccine a given subject should receive can be determined based on the genotype of the current viral stains in a population. For example, current viral isolated can be classified by the number of polymorphisms that they have. In one embodiment, the polymorphisms are ones that have been identified in isolates from pervious outbreaks. The identification of sequence polymorphisms in a population of viral isolates can be used to form an exposure timeline. This time line can be used to determine the age group susceptibility to a viral infection. For example, a new isolate with a number of polymorphisms identified in 1970 may be less of a concern to those people born prior to 1970, whereas this same isolate may produce more severe infection in those subjects born after 1970. Based on this timeline, medical professionals can determine which subjects should be administered a vaccine, or what vaccine a given subject should receive.

In another aspect, the invention provides a method of identifying the stability of a genome in a population of viruses, comprising: obtaining the nucleic acid or polypeptide sequence of one or more viral genes from a sufficient number of isolated viruses from the population; comparing the number of recombinant viral sequences in the isolated viruses; wherein the greater the number of distinct viral sequences, the greater the instability of the viral genome.

In another aspect, the invention provides a method of identifying the stability of a genome in a population of viruses, comprising: comparing the nucleic acid or polypeptide sequence of one or more viral genes from a sufficient number of isolated viruses from the population; comparing the diversity between parental viral sequences in the isolated viruses; wherein the greater the diversity of distinct viral sequences, the greater the instability of the viral genome.

Genetic stability can be used to measure environmental or experimental effects on genetic stability. This measurement can be determined actively or passively. Thus animals can be immunized and then co-infected with two parental strains and the progeny can be monitored to see the amount of recombination that occurs. This approach can be used to measure the ability of a vaccine to reduce or eliminate recombinants. Similarly, assaying a natural population at different time points can be used to measure environmental effects on recombination. The amount of genetic stability (or instability) can be used to identify times when aggressive intervention is necessary, even in the absence of overt disease.

In another aspect, the invention provides a method of immunizing a subject (e.g., a human or animal) against a virus comprising: administering to the subject drug resistant viral strains, or antigens or portions of antigens therefrom, made by recombination according to a copy-choice mechanism of two viral strains whose genomes are made up of non-identical nucleic acid sequences.

In another aspect, the invention provides a method of immunizing a subject (e.g., a human or animal) against a virus comprising: determining the parental viral strains in a population of viruses; allowing the parental viral strains to recombine according to a copy-choice mechanism to produce drug resistant viral strains; administering the parental viral strains, or drug resistant viral strains, or antigens or portions of antigens therefrom, in an amount sufficient to immunize the subject.

In another aspect, the invention provides a method for identifying parental influenza A and/or influenza B strains in a population of influenza viruses, wherein the population comprises parental influenza A and/or influenza B strains and drug resistant influenza A and/or influenza B strains, comprising the steps of: obtaining the nucleic acid or polypeptide sequence of one or more influenza A and/or influenza B genes from a number of isolated influenza A and/or influenza B strains from the population, the number sufficient to allow for identification of the influenza A and/or influenza B strains most prevalent in the population, the influenza A and/or influenza B strains having the greatest sequence divergence in the population, or both; identifying the influenza A and/or influenza B strains most prevalent in the population, or influenza A and/or influenza B strains with the greatest sequence divergence in the population, or both; wherein the most prevalent influenza A and/or influenza B sequences, or the influenza A and/or influenza B sequences with the greatest divergence are the parental influenza A and/or influenza B strains.

In a related embodiment, the invention provides a method of producing an influenza A and/or influenza B vaccine, comprising: infecting a host animal with the parental influenza A and/or influenza B strains identified; and isolating drug resistant influenza A and/or influenza B strains from the host animal.

In a related embodiment, the invention provides a method of immunizing a subject against an influenza A and/or influenza B virus comprising: administering to the subject a first influenza A and/or influenza B virus representing the first parental influenza A and/or influenza B strain and a second influenza A and/or influenza B virus representing a second parental influenza A and/or influenza B strain, the first and second parental influenza A and/or influenza B strains identified according to the methods described herein, in an amount sufficient to immunize the subject.

In one aspect, the invention provides a method of producing a library of recombinant viral strains comprising: infecting a host cell or animal with two or more viral strains; allowing for recombination of the viruses by a copy choice mechanism of the two or more viral strains, thereby creating a library of viral strains. In one embodiment, the library of recombination viral strains can be isolate for vaccine production.

In a related embodiment, the viral strains may be different species of viruses. For example, the first virus could be influenza A and/or influenza B and the second virus could be a coronavirus, e.g., SARS. In a related embodiment, the identification of a DNA sequence from one species' genome that originated in the genome of a distinct species is indicative that this segment of DNA confers an advantageous property to the virus, i.e., increased infectivity or virulence. Targeting these regions of DNA would provide for effective anti-viral therapy.

In a related embodiment, the library of viral strains can be created in a host cell or animal that has been given an antiviral compound. In a related embodiment, the viral strains that are created in the presence of an antiviral compound are indicative of the antiviral resistant strains that will occur in a population of subjects treated with the antiviral compound.

In another aspect, the invention provides a vaccine composition, comprising drug resistant influenza A and/or influenza B strains, or antigens or portions of antigens therefrom, made by recombination according to a copy-choice mechanism of two influenza A and/or influenza B strains whose genomes are made up of non-identical nucleic acid sequences.

In other embodiments, art-recognized methods of gene therapy, e.g., RNAi, may be employed to target viral strains, optionally in a strain and/or otherwise sequence-specific manner, e.g., via use of miRNA, siRNA, shRNA, or other such agents.

In another aspect, the invention provides a method for identifying parental coronavirus strains in a population of coronavirus viruses, wherein the population comprises parental coronavirus strains and drug resistant coronavirus strains, comprising the steps of: obtaining the nucleic acid or polypeptide sequence of one or more coronavirus genes from a number of isolated coronavirus strains from the population, the number sufficient to allow for identification of the coronavirus strains most prevalent in the population, the coronavirus strains having the greatest sequence divergence in the population, or both; identifying the coronavirus strains most prevalent in the population, or coronavirus strains with the greatest sequence divergence in the population, or both; wherein the most prevalent coronavirus sequences, or the coronavirus sequences with the greatest divergence are the parental coronavirus strains.

In a related embodiment, the invention provides a method of producing a coronavirus vaccine, comprising: infecting a host animal with the parental coronavirus strains identified; and isolating drug resistant coronavirus strains from the host animal. In another aspect, the invention provides a method of immunizing a subject against an coronavirus virus comprising: administering to the subject a first coronavirus virus representing the first parental coronavirus strain and a second coronavirus virus representing a second parental coronavirus strain, the first and second parental coronavirus strains identified according to the methods described herein in an amount sufficient to immunize the subject.

In one aspect, the invention provides a vaccine composition, comprising drug resistant coronavirus strains, or antigens or portions of antigens therefrom, made by recombination according to a copy-choice mechanism of two coronavirus strains whose genomes are made up of non-identical nucleic acid sequences.

In another aspect, the invention provides a method of producing drug resistant viral strains for the manufacture of a viral vaccine comprising; infecting a cell or animal with two non-identical viral strains; allowing for recombination of the non-identical viral strains according to a copy-choice mechanism; thereby producing drug resistant viral strains. In one embodiment, the method further comprises isolating the drug resistant viral strains from the host cell or animal.

In one aspect, the invention provides a method of determining the efficacy of a vaccine comprising: obtaining the nucleic acid or polypeptide sequence of one or more viral genes from a number of isolated viral strains from a population that has been treated with a viral vaccine, the number sufficient to allow for number of drug resistant viral strains in the population; wherein, the lower the number of different drug resistant viral strain sequences, the greater the efficacy of the vaccine.

In another embodiment, the invention provides a method of predicting the sequence of one or more genes in a drug resistant viral strain comprising obtaining the sequence of one of more of the genes from a parental viral strain, determining the location of possible recombination events, thereby predicting the sequence of one or more genes in a drug resistant viral strain. In a related embodiment, the viral strain is selected from the group consisting of an influenza A and/or influenza B viral strain, a corona viral strain, and an HIV viral strain. In another related embodiment, the method further comprises using the predicted sequence of the drug resistant viral strain to develop a vaccine against said virus.

In another aspect, the invention provides a method of producing drug resistant viral strains comprising infecting a cell or animal with two non-identical viral strains, allowing for recombination of the non-identical viral strains according to a copy-choice mechanism, thereby producing drug resistant viral strains. In a related embodiment, the method further comprises isolating said drug resistant viral strains.

In a related aspect, the invention provides a method of producing drug resistant virus(es) comprising infecting a cell or animal with two or more non-identical viruses (e.g., ebola and influenza A or influenza B), allowing for recombination of the non-identical viruses according to a copy-choice recombinant mechanism, thereby producing drug resistant virus(es). In related embodiments, the method further comprises isolating and/or raising vaccine(s) to said drug resistant virus(es).

In a related aspect, the invention provides a method of predicting a phenotypic trait (e.g., virulence, drug resistance, etc.) of a drug resistant progeny virus, bacteria or plant through assessment of the range of drug resistant possible via copy-choice recombination from two or more parental viruses, bacteriae or plants.

In another aspect, the invention provides a method of producing a population of recombinant genes comprising introducing into a cell two or more non-identical copies of a gene, allowing for recombination of the genes, thereby producing a population of recombinant genes. In a related embodiment, the recombination occurs via a copy-choice mechanism. In a related embodiment, the method further comprises isolating one or more members of the population of recombinant genes. In one embodiment, the genes are viral genes. In another embodiment, the genes are from non-viral species, e.g., plants or animals.

In certain aspects, the present invention concerns the genetic transfer of polymorphic sites between strains of influenza. Within the influenza genome, sites of clinical relevance to humans are predicted to be those that enhance the molecular specificity, infectivity, virulence, propagation, etc. of influenza virus within a human subject, as compared to, e.g., an avian subject. An exemplary mutation documented to increase the affinity of the HA protein of H5 strains of virus for human glycoprotein receptors, as compared to avian glycoprotein receptors, is the S227N polymorphism (H3 residue numbering used; by H5 residue numbering, termed the S223N polymorphism) featured in certain embodiments of the present invention (Hoffmann et al., Proc. Natl Acad. Sci. 102: 12915-20). Additional polymorphisms within the influenza genome predicted to impart, e.g., molecular specificity, infectivity, virulence, propagation, transmission, etc., of heightened human impact to the influenza virus include documented polymorphisms in HA (e.g., mutations at residue(s) 190, 225, 226, 227 (e.g., S227N) and/or 228 (G228S) (H3 residue numbering) and/or residue(s) 36, 83, 86, 120, 155, 156, 189, 212, 263 (H5 residue numbering)), PB2 (e.g., mutations at residue(s) 627 (e.g. , E627K, shown to be important to mammalian adaptation of the 1918 pandemic influenza virus), 199 (e.g., A199S), 475 (e.g., L475M), 567, 627 and /or 702), PB1 (e.g., mutation(s) at residue(s) 54 (e.g., K54R), 375, 383, 473, 576, 645 and/or 654), and PA (e.g., mutations at residue(s) 241 (e.g., C241Y), 312 (e.g., K312R), 322 (e.g., I322N), 55 (e.g., D55N), 100 (e.g., V100A), 312, 322, 382 (e.g., E382D) and/or 552 (e.g., T552S)) (Hoffmann et al., Proc. Natl Acad. Sci. 102: 12915-20; Taubenberger et al., Nature 437: 889-93; Gambaryan et al., Virology 344: 432-38; Stevens et al., J. Mol. Biol 355: 1143-55). Additional exemplary mutations identified in the influenza genome and of potential human impact are presented in Table 21.

Both known and newly-identified mutations in the influenza genome can be tested for their potential impact on human molecular specificity, infectivity, virulence, propagation, transmission, etc., via art-recognized methods (e.g., propagation of influenza in, e.g., Vero or MDCK cells, as compared to chicken embryo cells; molecular modeling approaches to identify potential impact of mutations upon, e.g., HA binding to receptors and/or impact of mutants upon function of the heterotrimeric polymerase complex (PA, PB1, PB2)). In certain embodiments of the present invention, the molecular affinity of the HA protein of influenza for specific receptor glycoproteins (e.g., glycoproteins more prevalent in the human respiratory tract, such as a-2-6-linked sialic acids, as compared to glycoproteins more common in the avian enteric tract) is directly assayed in vitro via use of glycan microarrays. Glycan microarrays as described in Stevens et al. (J. Mol. Biol. 355: 1143-55) allow for rapid assessment of the impact of any mutation in the HA protein of influenza upon the affinity of HA for an extensive panel of glycan modifications, a selection of which are more prevalent in the mammalian respiratory tract. Within the present invention, assay of mutant HA proteins for glycan specificity can be performed on either parental strains of virus (e.g., to prioritize geographic tracking of specific mutation(s) of heightened predicted, e.g., human/clinical impact, on the basis of an observed glycan microarray binding profile) or progeny strains of virus (e.g., to perform an in vitro assessment of specific progeny strains of virus predicted to arise from two or more parental strains of virus). Details regarding performance of such assays can be found in Stevens et al., the contents of which are incorporated herein by reference in their entirety.

Certain aspects of the present invention involve the mixing of two or more parental strains of virus for purpose of ascertaining the identity of drug resistant strains of virus arising therefrom. While such mixing experiments can be modeled by hand and/or in silico, physical mixing of parental strains can be performed either in vitro or in vivo by art-recognized approaches of propagating influenza virus. For example, in vitro mixing of parental strains of influenza virus can be performed in a wide range of cell types, including chicken embryonic cells, and a number of mammalian cell lines, e.g., Vero (derived from African green monkey kidney) and MDCK (canine kidney) cells (refer to Mochalova et al., Virology 313: 473-80). In certain embodiments of the present invention, performance of such parental strain mixing experiments in mammalian cell lines, particularly primate cell lines, is preferred, for purpose of selecting in favor of viral strains more likely to impact human specificity, propagation, virulence, infectivity, etc. (in certain instances, propagation of influenza in e.g., chicken embryo cells, might be anticipated to select away from human/primate-specific strains of virus, potentially limiting the information to be gained via performance of mixing experiments in such cells). Accordingly, the viral strain mixing experiments of the present invention may be performed in any art-recognized cell line capable of propagating the influenza virus (refer to “Influenza Vaccine Production” section below).

In certain embodiments of the present invention, mixing of parental viral strains can also be performed in vivo. For example, avian and/or mammalian host organisms can be infected with parental strains of virus (including attenuated strains of virus) in order to discern the identity of specific progeny strains of virus arising from such combined infection of host organisms with the parental strains. Host organisms can include any avian and/or mammalian organism, including, e.g., mammals, e.g., primates, dogs, cows, horses, swine, sheep, goats, cats, mice, rabbits, rats, and transgenic non-human animals, or birds, e.g., ducks, chicken, geese, turkeys, quail and swans. Two parental viral sequences can be combined in vitro, in vivo, or in silico, with the rules of the present invention allowing for enhanced prediction of which drug resistant virus(es) will exhibit a monitored trait. The present invention can therefore be applied, e.g., to drug screening approaches, vaccine production, diagnostic (kit) production, etc. It is understood that zoonotic dieoffs (e.g., ducks, swans, quail, swine), especially in particular geographic areas, can be used to predict parental strains that will contribute to progeny strains of virus via the gene transfer events of certain aspects of the present invention.

It is understood that the invention also encompasses the application of predicting the emergence of influenza strains from sequences derived from domestic and/or farm animals (e.g., swine isolate sequences). As set forth in the Examples below, such animals, e.g., swine, may act as a sequence reservoir over longer spans of time than are normally seen in the evolution of rapidly spreading migratory bird and human strains of influenza. Such sequence reservoirs may then be drawn upon via recombination with, e.g., migratory bird and/or human sequences, contributing as parental strains to future progeny strains of influenza.

As also set forth in the Examples below, mapping of parental strains through use of appropriate probe sequences to individual influenza haplotypes can reveal the transition of a sequence from, e.g., an H1 strain to a more aggressively virulent H5 strain. Observation of such strain-transitional flow of influenza sequence can reveal polymorphic sequences of particular importance for vaccine development against future progeny strains of influenza.

### Influenza Vaccine Production

Certain embodiments of the invention involve production of vaccines to, e.g., progeny viral strain sequences of the invention. The generation of such vaccines can be performed by any art-recognized method. Exemplary methods of vaccine production involve production/propagation of virus, purification and formulation of virus and/or viral components for use as vaccines, and administration of such vaccines.

Viral production systems known in the art include, e.g., those described in U.S. Pat. Nos. 6,544,785; 6,649,372 (featuring methods for generating in cultured cells (e.g., Vero cells) infectious viral particles of a segmented negative-strand virus without using helper virus, including vaccines and compositions produced by such methods); U.S. Pat. No. 6,146,642 (featuring a recombinant RNA molecule comprising a binding site specific for an RNA-directed RNA pol of a Newcastle disease virus (NDV)), linked to a viral RNA containing a heterologous RNA sequence; U.S. Pat. No. 6,669,943 (featuring an attenuated influenza virus with modified NS1 gene and interferon antagonist phenotype, including vaccines and pharmaceutical formulations made therefrom); U.S. Pat. No. 6,573,079 (featuring methods of vaccine production via propagation of an attenuated influenza virus having a mutation in the NS1 gene that reduces the cellular interferon response); U.S. Pat. No. 5,989,805 (featuring methods for propagating and/or preparing an avian virus, e.g., influenza, using chicken embryonic cells); U.S. Pat. No. 5,948,410 (featuring influenza surface antigen vaccines from influenza virus propagated in animal cells (e.g., canine kidney cells (MDCK)) and substantially free of host cell DNA); U.S. Pat. No. 4,552,758 (featuring a method of preventing influenza A virus in humans; U.S. Pat. No. 4,552,757 (featuring an influenza vaccine for use in non-avian animals, the vaccine comprising NP or M proteins from specified strains); U.S. Pat. No. 6,344,354 (featuring a vaccine comprising a replicated mammalian influenza virus passaged in cells that are not eggs, including methods of vaccination using the same); U.S. Pat. No. 5,824,536 (featuring methods of making vaccine via infection of mammalian cells (e.g., Vero cells) and culturing in trypsin-containing media); U.S. Pat. Nos. 6,048,535; 6,406,702 (featuring multivalent poultry vaccines safe for ovo inoculation comprising the agents Newcastle disease virus (NDV) and, e.g., influenza virus, also featuring vaccine methods using such vaccines); U.S. Pat. No. 6,322,967 (featuring a method of making influenza virus with a modified PB2, an influenza virus made according to such a method, and a method of treating humans with such a vaccine); U.S. Pat. No. 6,146,873 (featuring methods for producing orthomyxovirus (influenza) virus using monkey kidney cells in protein-free media); U.S. Pat. No. 5,753,489 (featuring the methods of U.S. Pat. No. 6,146,873, wherein cells are instead adapted to serum-free media); U.S. Pat. No. 4,500,513 (featuring methods of preparing influenza vaccine using cell culture and a proteolytic enzyme (e.g., trypsin)); U.S. Pat. No. 5,756,341 (featuring methods of producing influenza vaccine antigens in serum-free cell culture using HA with a modified cleavage site); and U.S. Pat. No. 5,698,433 (featuring methods of preparing influenza virus using avian embryo cells and a serine protease). The preceding U.S. Patents are incorporated in their entirety herein by reference.

Vaccine purification and formulation methods and compositions described in the art include, e.g., U.S. Pat. No. 6,060,068 (featuring a vaccine (e.g., for equine influenza) that comprises IL-2 as a coadjuvant); U.S. Pat. No. 6,451,325 (featuring an influenza virus vaccine formulation comprising metabolizable oil adjuvant); U.S. Pat. No. 5,709,879 (featuring an influenza virus vaccine formulation comprising metabolizable oil adjuvant in a liposome possessing net negative charge); U.S. Pat. No. 6,743,900 (featuring methods of preparing an influenza vaccine formulation using a proteosome preparation); U.S. Pat. No. 6,387,373 (featuring an influenza vaccine formulation comprising an oil-containing lipid adjuvant); U.S. Pat. No. 5,795,582 (featuring an influenza vaccine formulation comprising a dendrimer adjuvant); U.S. Pat. No. 5,919,480 (featuring an influenza vaccine formulated as a liposome comprising a cytokine, including methods of administration of same); U.S. Pat. No. 5,639,461 (featuring an influenza vaccine 99% inactivated by heat and formulated with thimerosal, including methods of administration of same); U.S. Pat. No. 3,919,044 (featuring methods of purifying and concentrating virus (e.g., influenza) using filtering and cationic exchange); U.S. Pat. No. 4,000,257 (featuring methods of extracting pyrogens and endotoxins from an influenza virus vaccine); U.S. Pat. No. 6,231,860 (featuring stabilizing agents (e.g., urea) for attenuated viral vaccines (e.g., influenza vaccine)); and U.S. Pat. No. 6,048,537 (featuring methods of preparing purified mixtures of influenza viral antigens by fragmenting live virus).

Methods of use and/or administration of anti-viral vaccines known in the art include, e.g., U.S. Pat. No. 5,916,879 (featuring a method of immunizing an avian with DNA encoding influenza H5); U.S. Pat. No. 5,643,578 (featuring methods of immunizing a vertebrate with DNA encoding HA of an infectious agent (e.g., influenza)); U.S. Pat. No. 6,159,472 (featuring a method of immunizing an avian intradermally with a vaccine comprising inactivated immunogen (though the vaccine can comprise, e.g., a live influenza immunogen)); U.S. Pat. No. 6,682,754 (featuring a method of inducing immunity via an implant comprising an immunogen (e.g., derived from influenza virus)); U.S. Pat. Nos. 5,817,320; 5,750,101 (featuring a method of ovo-immunization via administration of a vaccine into an egg air cell); U.S. Pat. No. 6,506,385 (featuring method of immunizing against avian viral disease via administration of live virus and interferon to an egg); and U.S. Pat. No. 5,149,531 (featuring a method of treating a subject (e.g., a bird subject) with a cold-adapted live influenza vaccine).

### Examples

The recent reports of high levels of osletamivir (neuraminidase inhibitor) resistance in seasonal influenza have caused concern because the resistance was in patients who had not recently taken osletamivir, and the genetic change, H274Y, was the same change that confers resistance to oseltamivir in the potential pandemic influenza sub-type, H5H1 (see, e.g., World Health Organization Collaborating Centers, Influenza A(H1N1) virus resistance to oseltamivir—2008/2009 influenza season, northern hemisphere, December 2008; Centers for Disease Control and Prevention—Korea, Antiviral resistant influenza A viruses isolated in Korea during 2008-2009 season—Oseltamivir resistance to A/H1N1 in Korea, January 2009; Lackenby, et al., Emergence of resistance to oseltamivir among influenza A(H1N1) viruses in Europe, Eurosurveillance, 13, Jan. 2008; and de Jong M D, et al. Oseltamivir resistance during treatment of influenza A (H5H1) infection. N Engl J Med 353,2667-2672 (2005), the entire contents of each of which are expressly incorporated herein by reference). Moreover, the resistance was specific for H1N1 and position H274Y, supporting the notion that the high levels was not linked to oseltamivir usage.

Prior studies of oseltamivir resistance in Japan were linked to sub-optimal dosing and resistance was found in both current influenza A subtypes, H1N1 and H3N2, and included, but was not limited to H274Y (see, e.g., Kiso, M K, et al., Lancet: 364,759-765 (2004), the entire contents of which are expressly incorporated herein by reference). Prior studies also supported a fitness penalty for the acquisition of H274Y, which predicted that the change would be limited to patients receiving oseltamivir (see, e.g., Ives et al., Antivir. Res: 55,307-317 (2002) and Herlocher M L et al., J Infect Dis:190,1627-1630 (2004), the entire contents of each of which are expressly incorporated herein by reference).

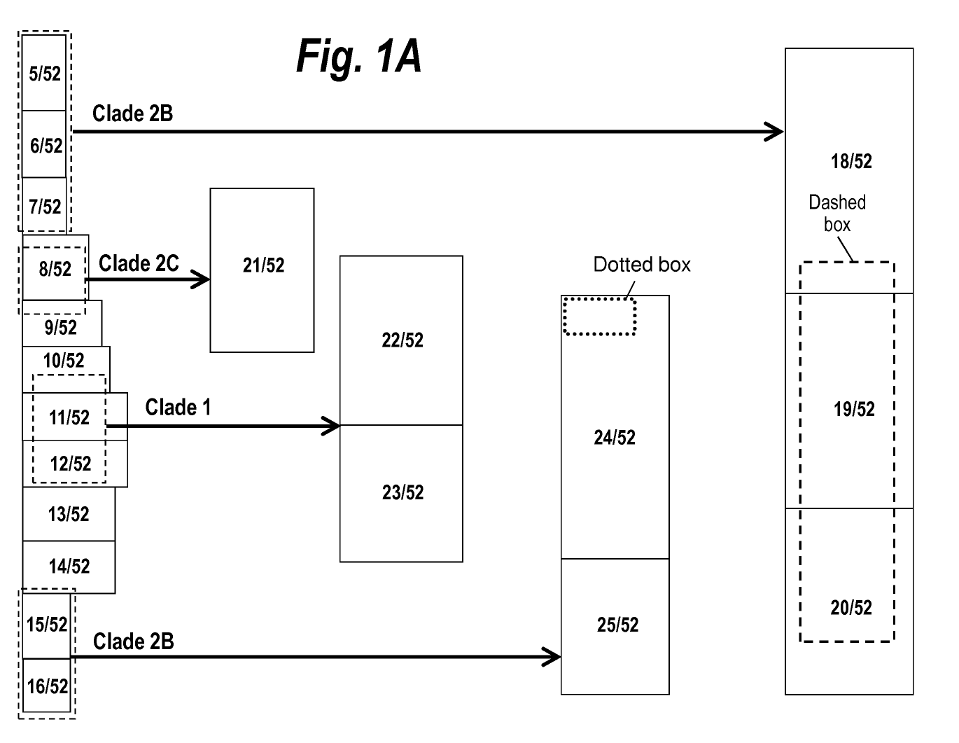

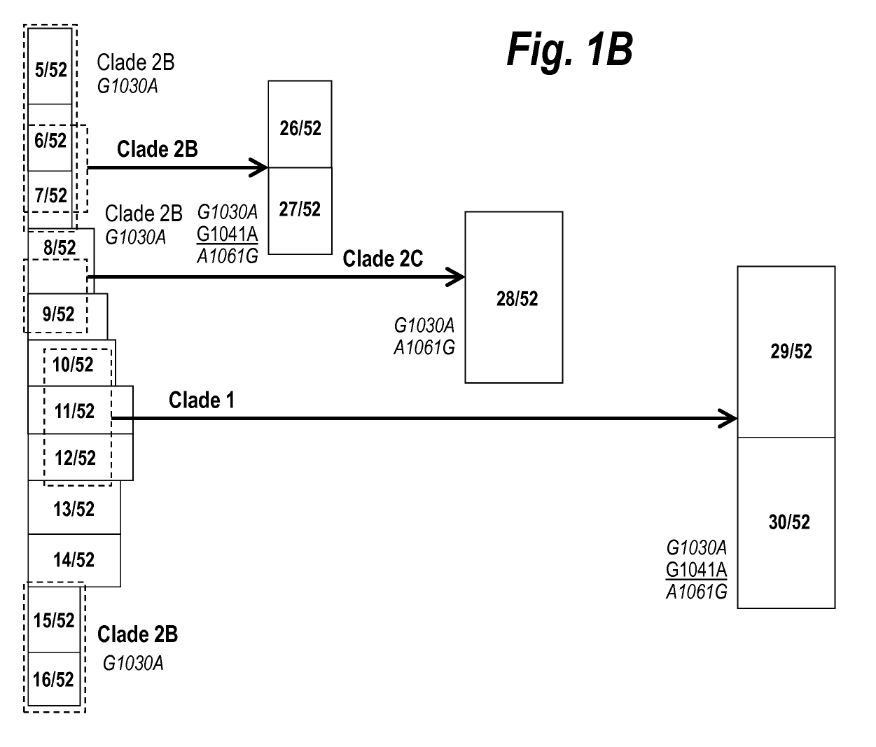

However, the appearance of H274Y in hosts not receiving oseltamivir was reported in wild birds infected with H5H1 in Astrakhan, which was followed by patients in China infected with H1N1 in 2006 (see, e.g., Sheu, et al., Antimicrob Agents Chemother;52,3284-3292 (2008), the entire contents of which are expressly incorporated herein by reference). Earlier H1N1 isolates had been closely related to the H1N1 vaccine target, isolated in New Caledonia in 1999 and designated Glade 1 (prototype New Caledonia/20/1999). Subsequent sub-clades were designated Glade 2 and divided into three sub-clades, 2A (prototype A/Solomon Island/3/2006), 2B (prototype A/Brisbane/59/2007) and 2C (prototype A/Hong Kong/2562/2006)—see the neuraminidase (NA) phylogenetic tree in FIG. 1. The patients from China demonstrated that the absence of a fitness penalty in the H5H1 wild birds extended to H1N1 seasonal flu, but the H274Y frequency remained low as seen in the multiple introductions in FIG. 1A.

In the 2006/2007 season H274Y appeared on another H1N1 genetic background, clade 1 (FIG. 1A). The clade 1 result was similar to clade 2C. Patients not taking oseltamivir were infected with oseltamivir resistant H1N1. However, the levels remained low even though the distribution on the phylogenetic tree supported multiple independent introductions, indicating that H1N1 with H274Y had not gained a significant selection advantage.

In the 2007/2008 season, H274Y appeared on yet another H1N1 genetic background, Clade 2B. The first reported isolates in the United States were in Hawaii (FIG. 1A dotted box), and this subclade was subsequently identified in Scotland and England, followed by France and Japan in 2008. However, the frequency of this sub-clade remained low. Subsequently, a much larger sub-clade emerged (FIG. 1A (Clade 2B* in dashed box).

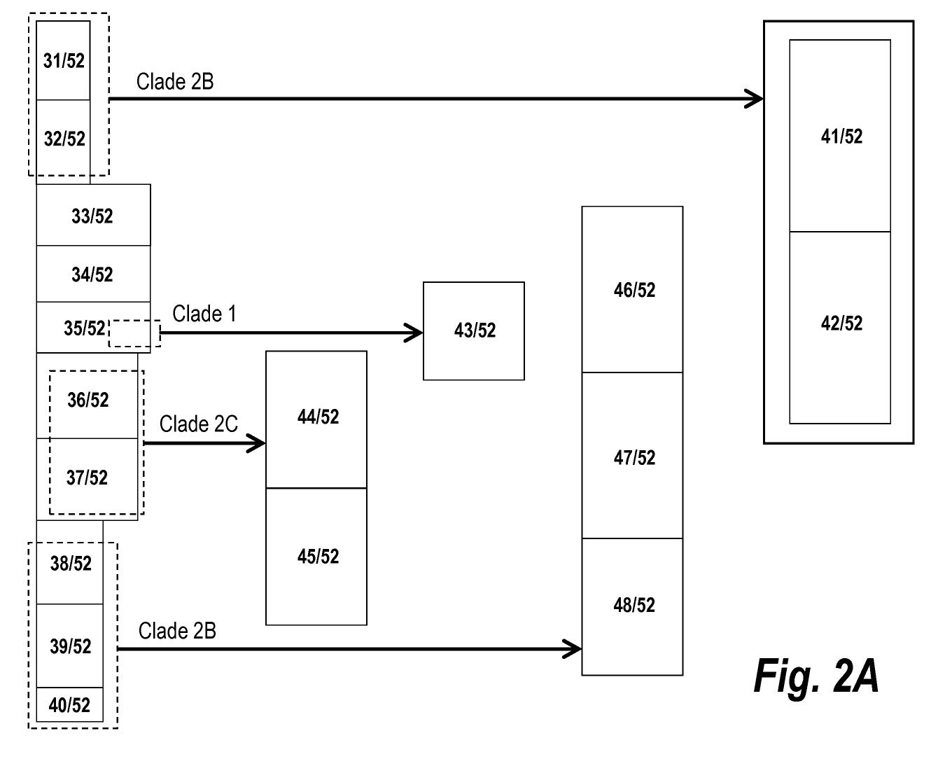

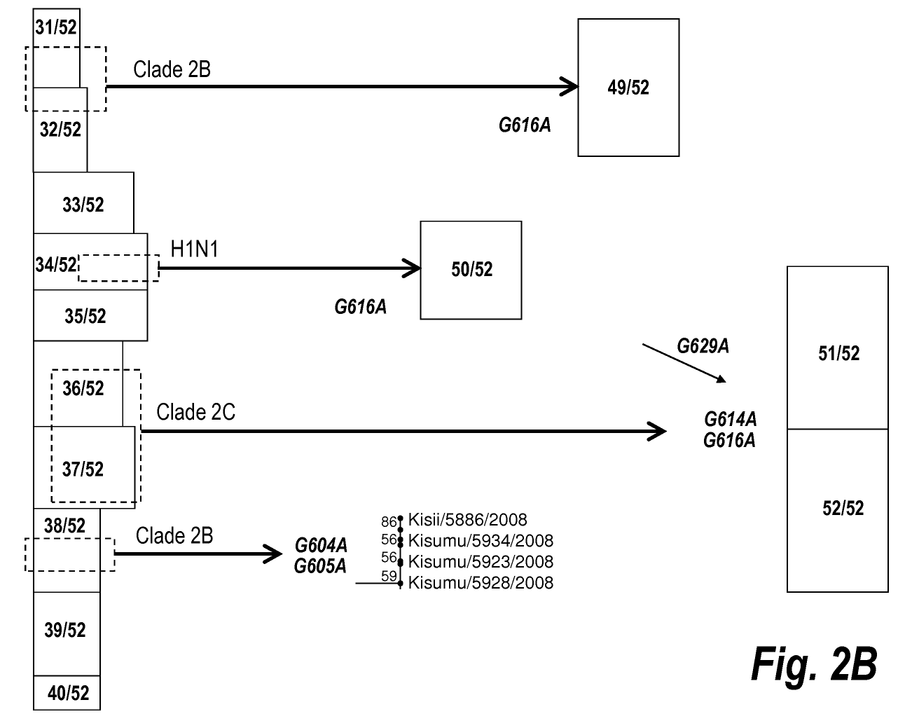

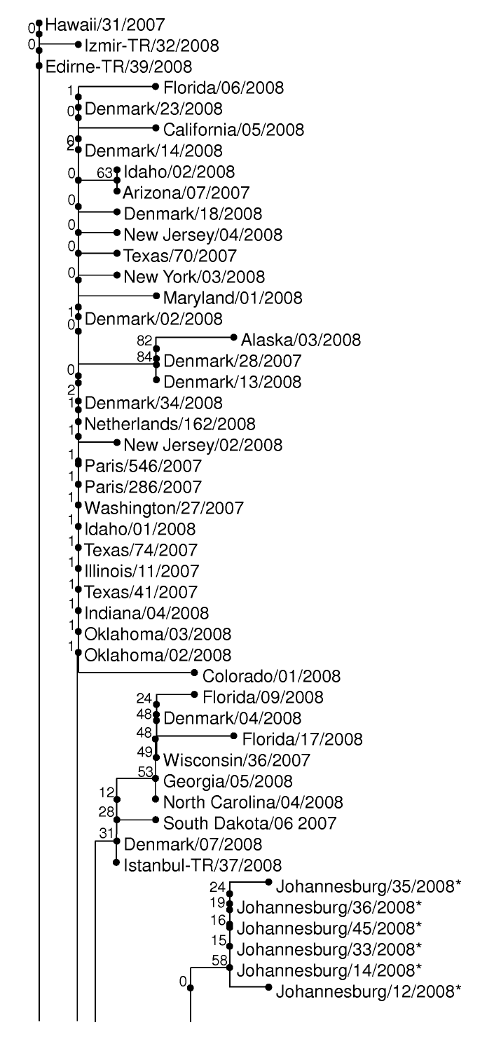

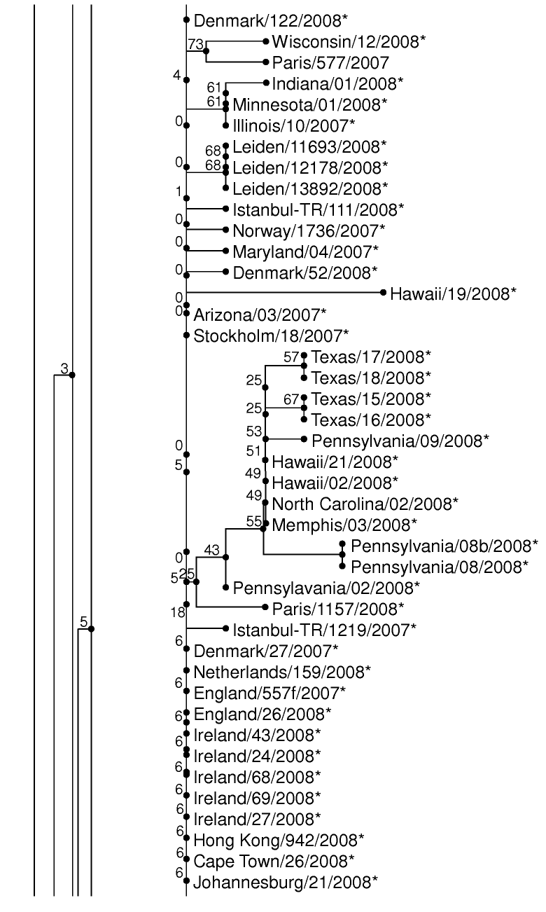

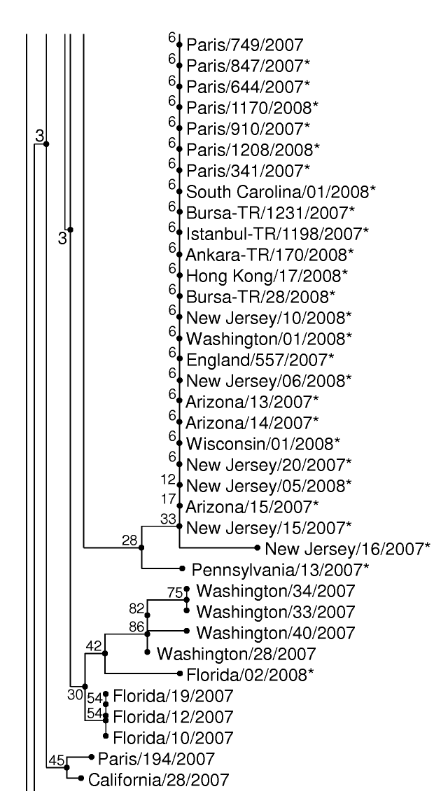

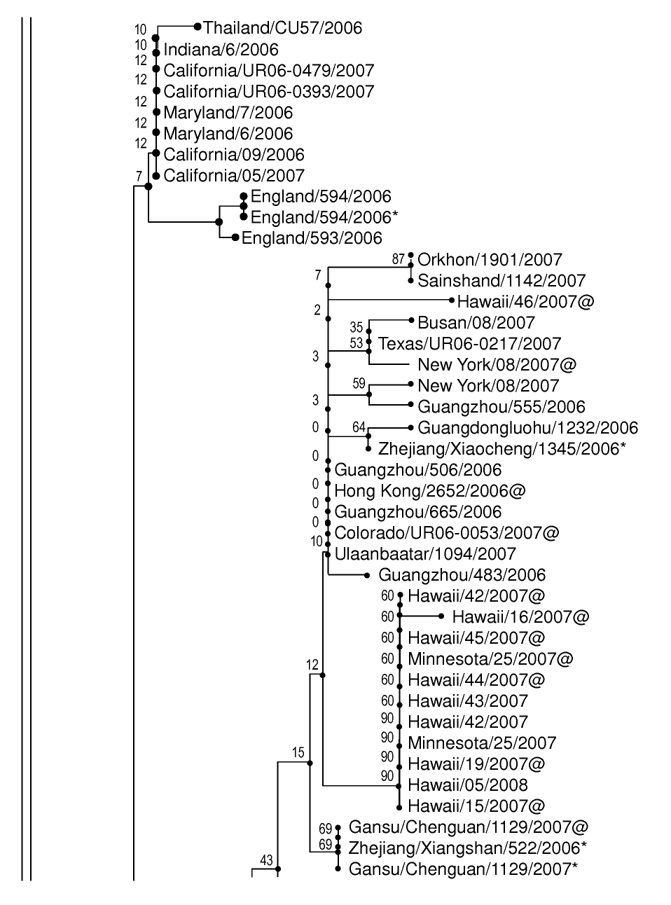

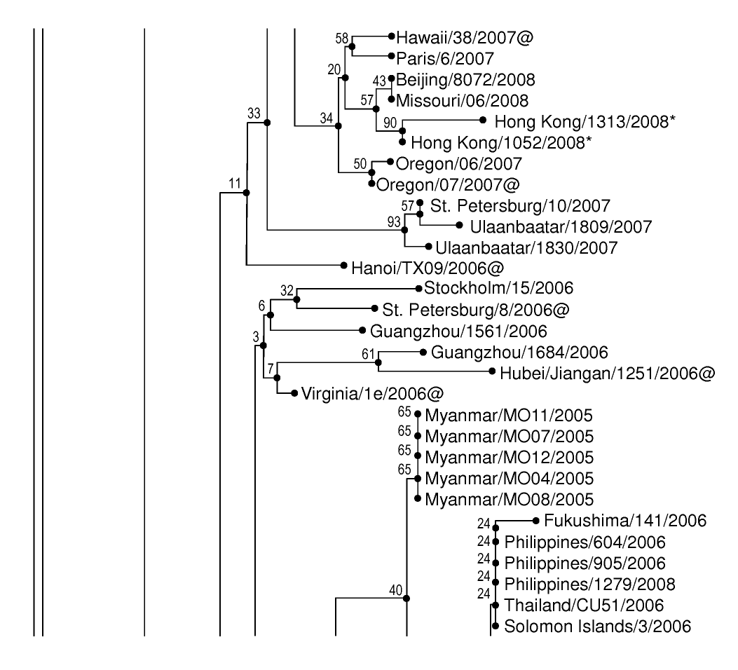

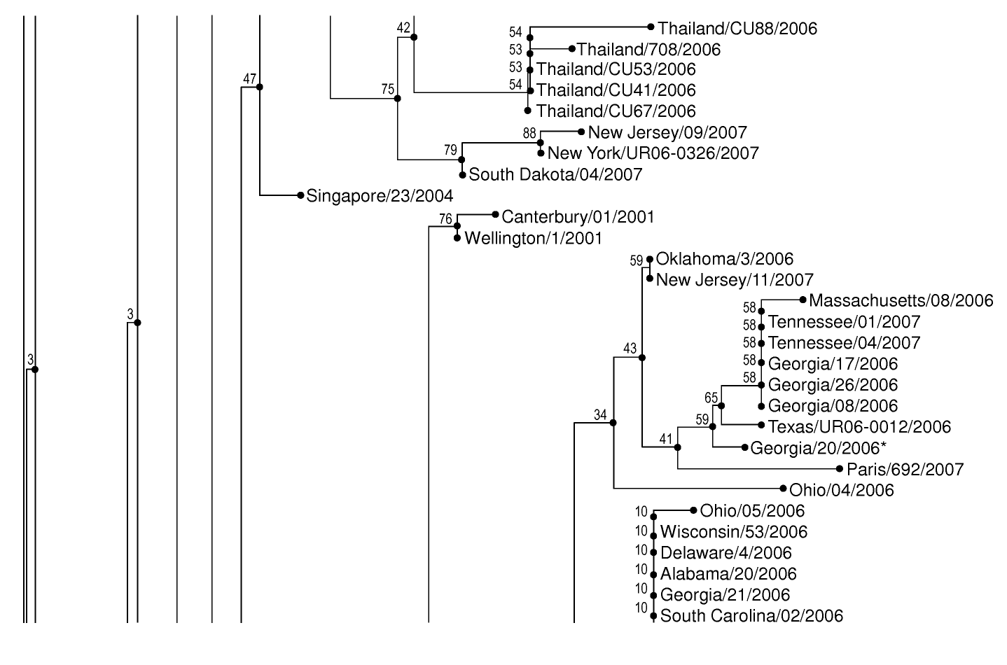

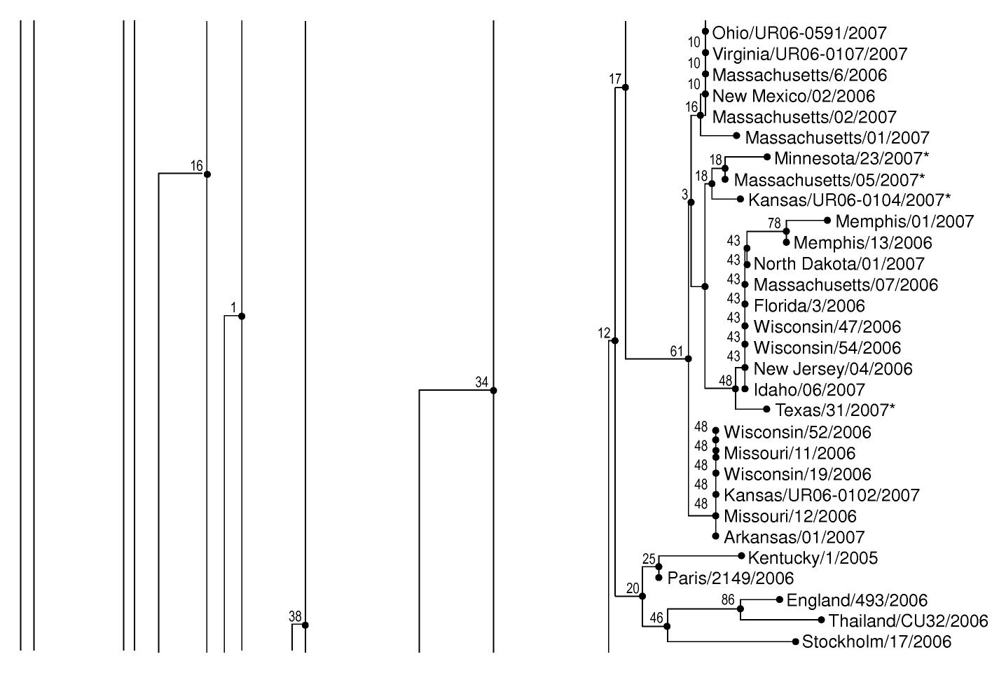

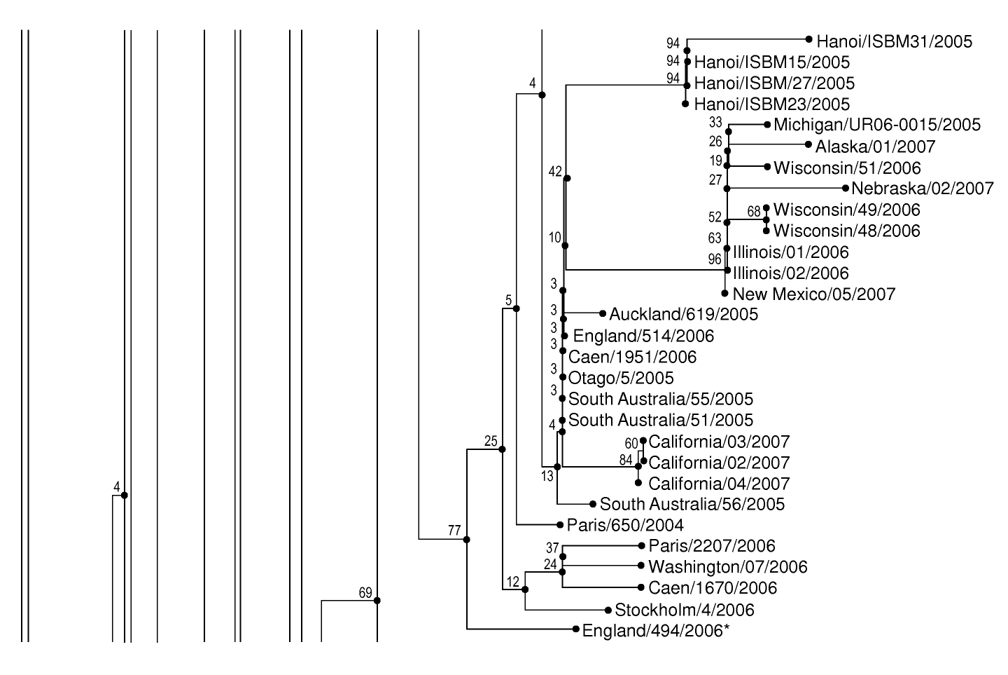

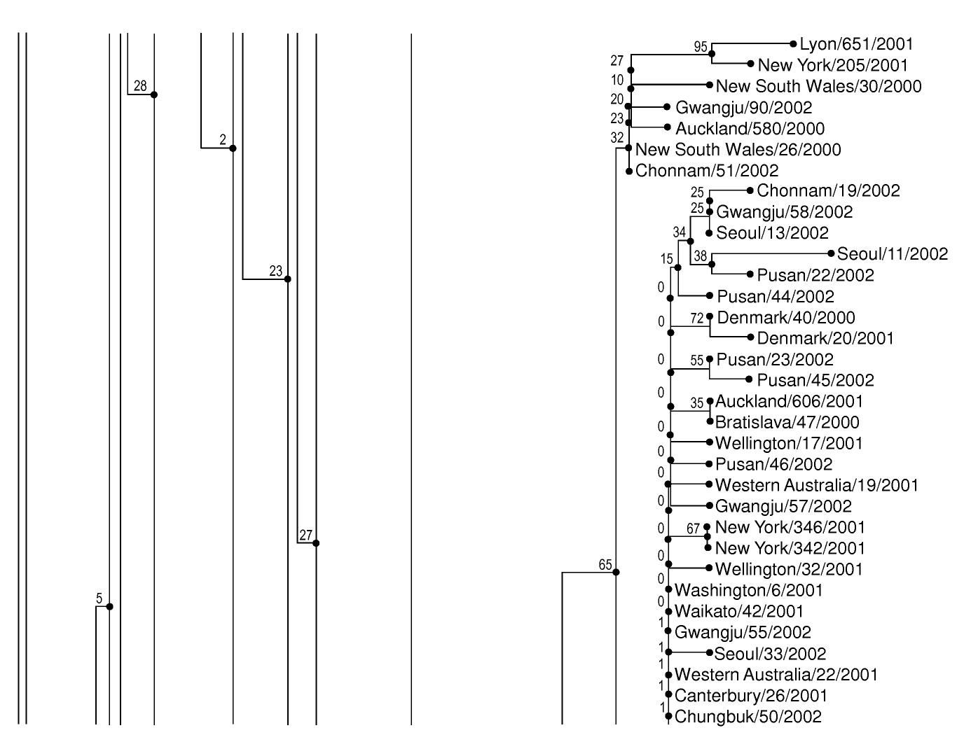

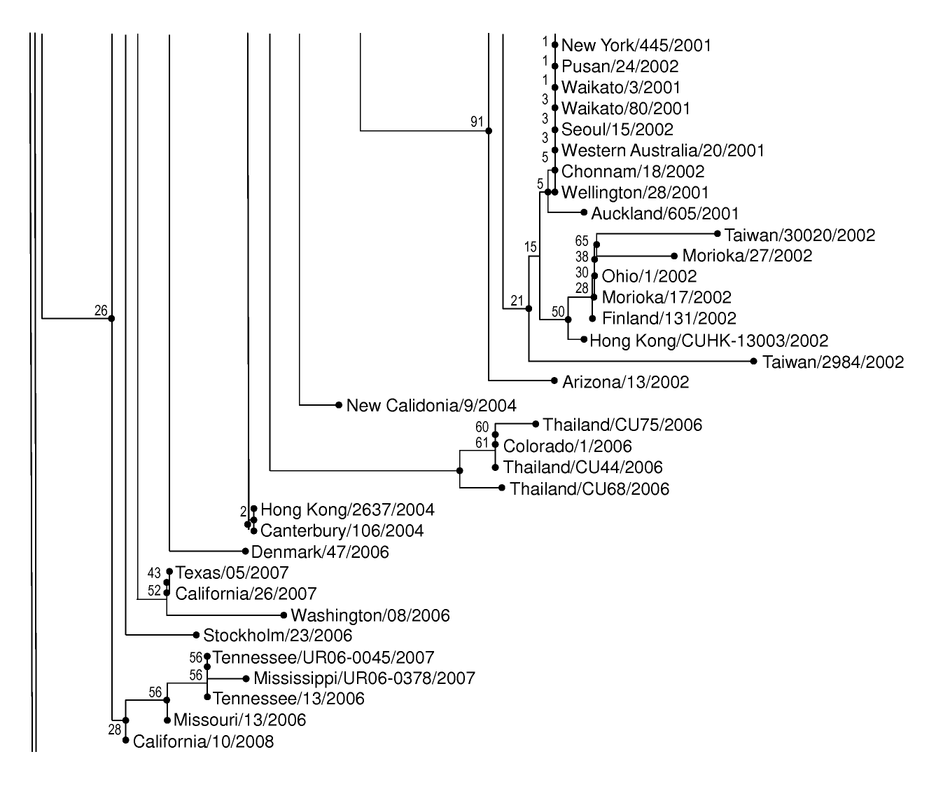

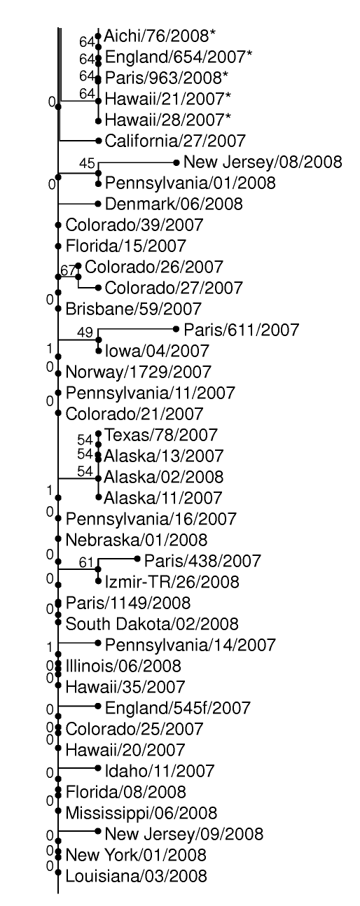

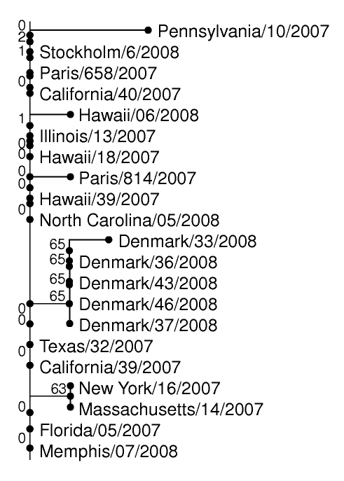

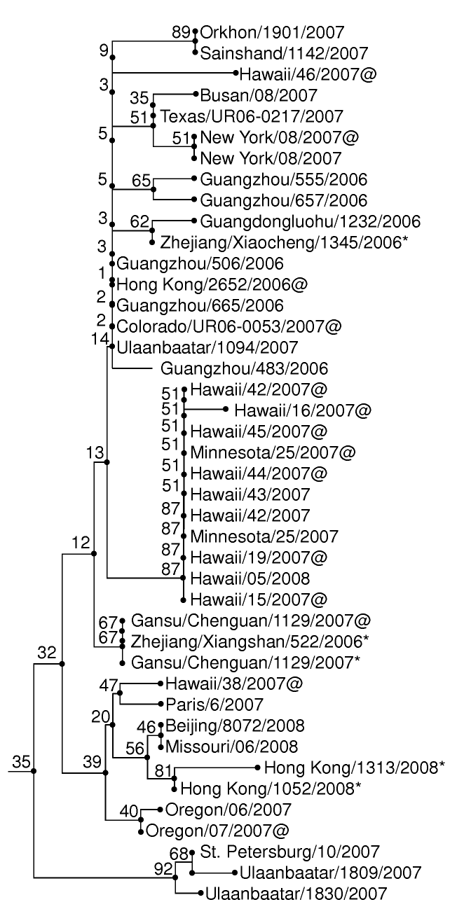

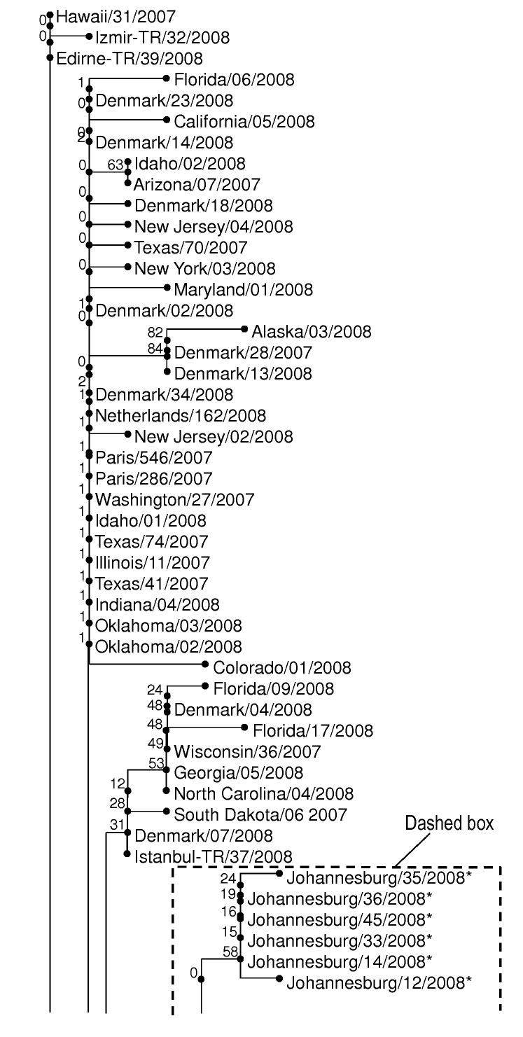

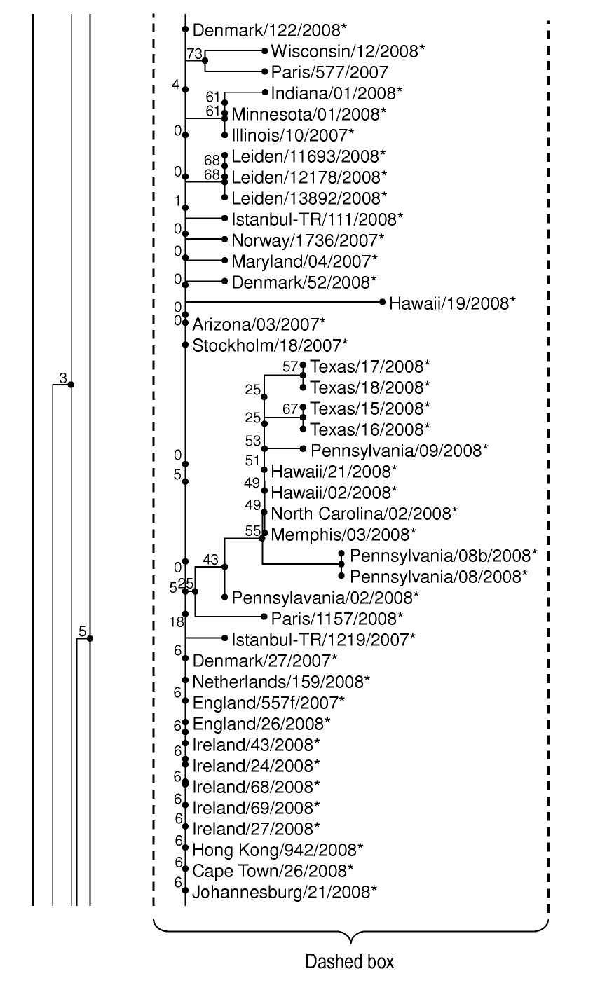

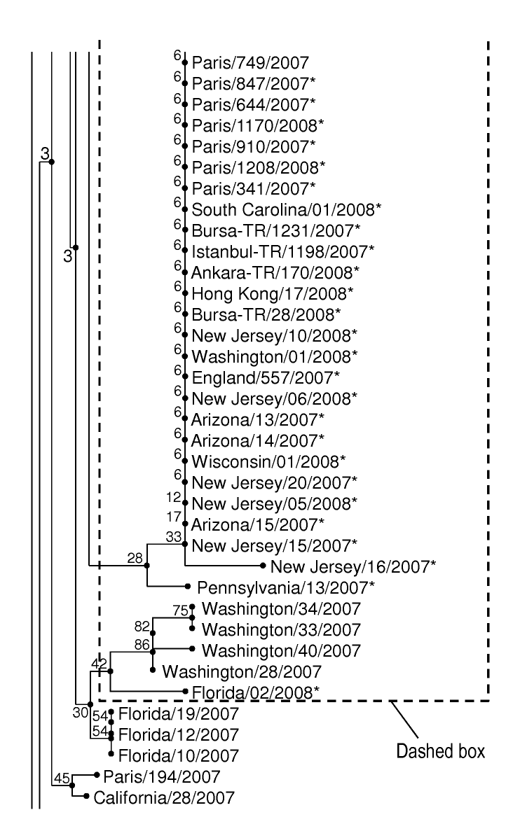

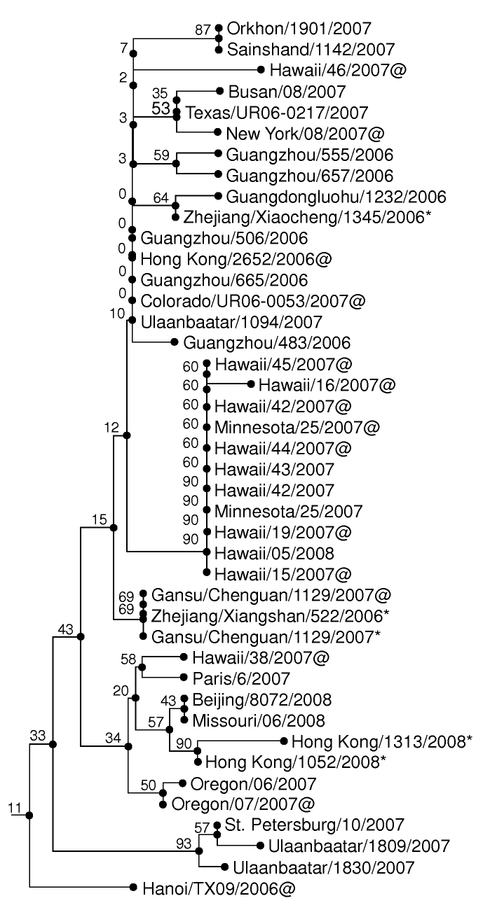

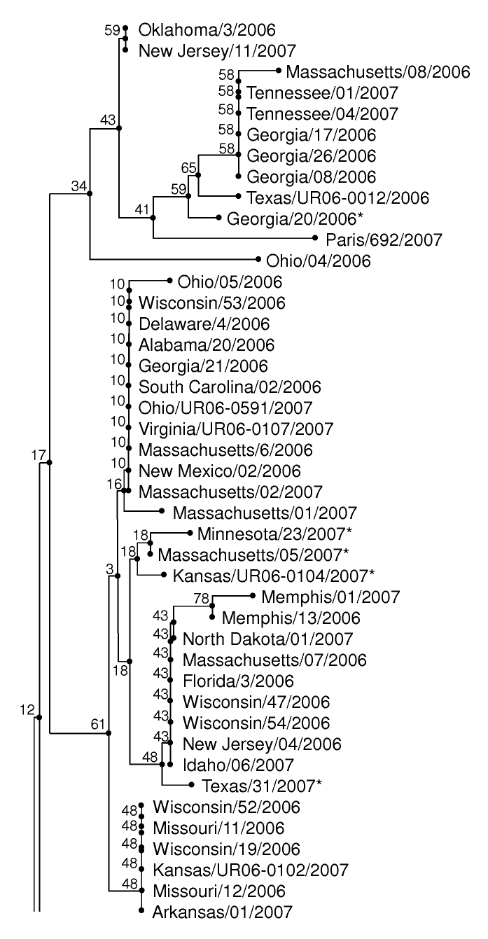

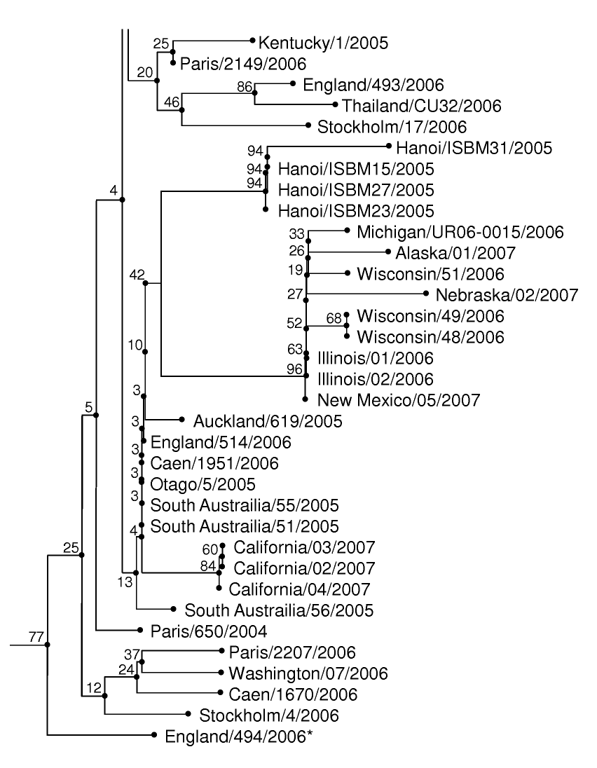

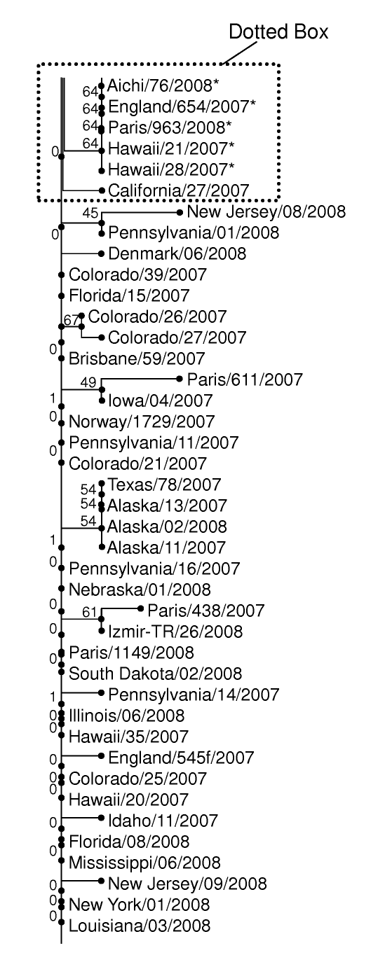

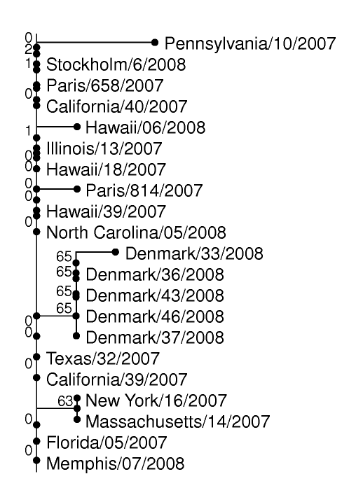

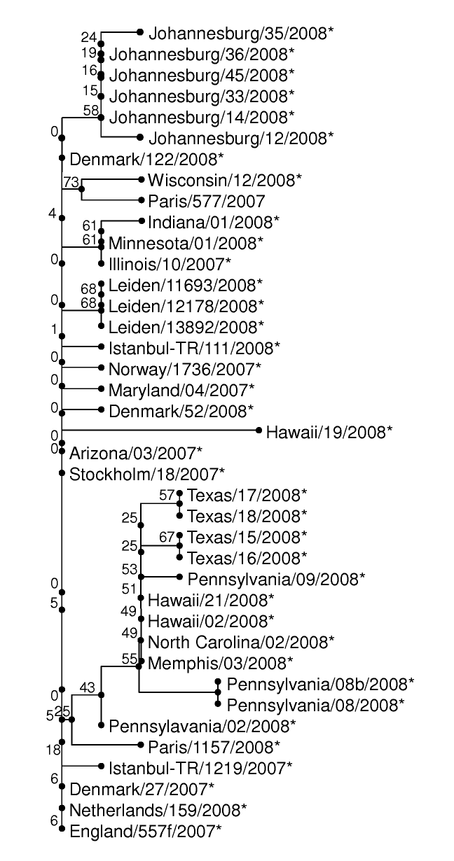

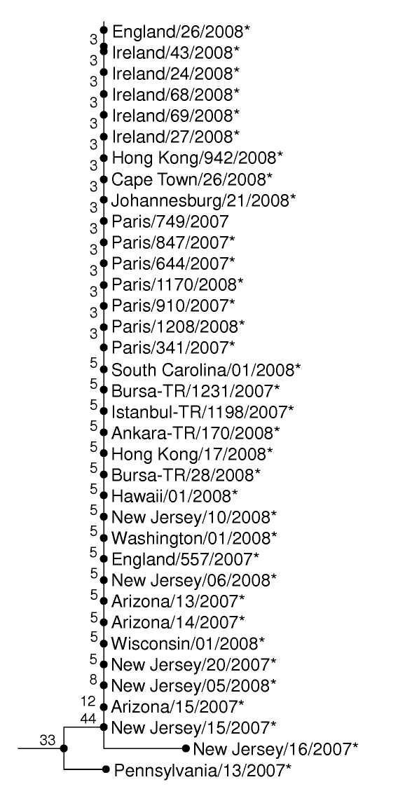

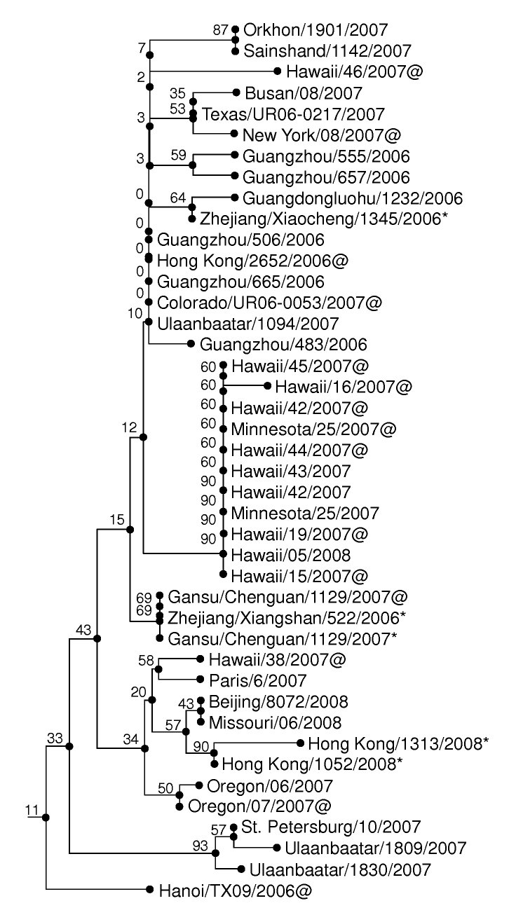

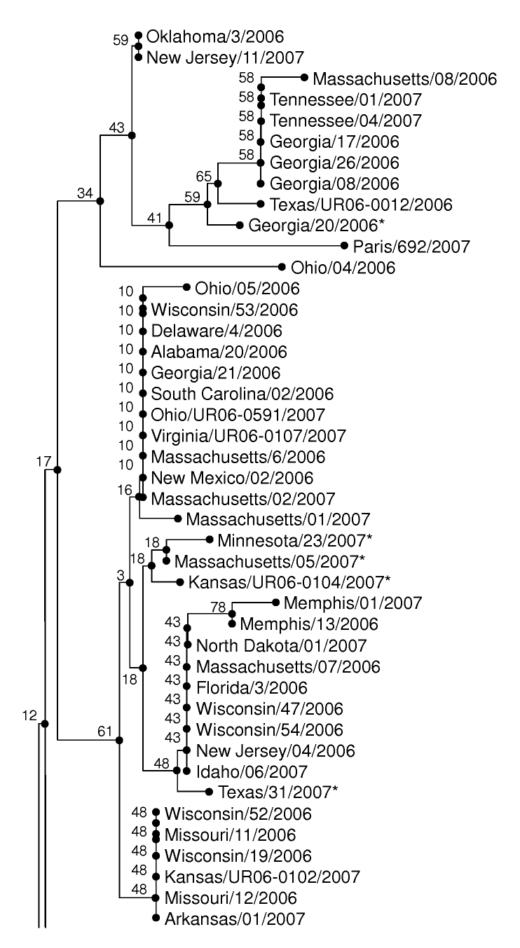

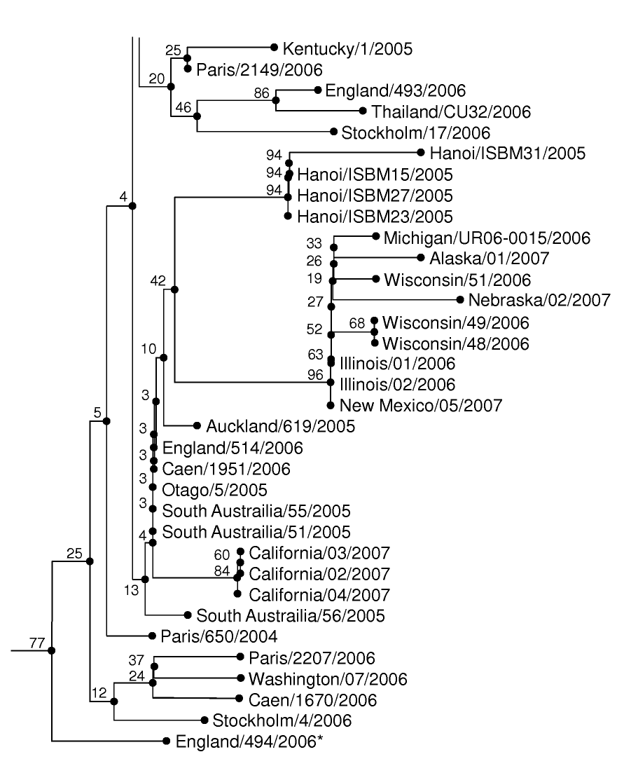

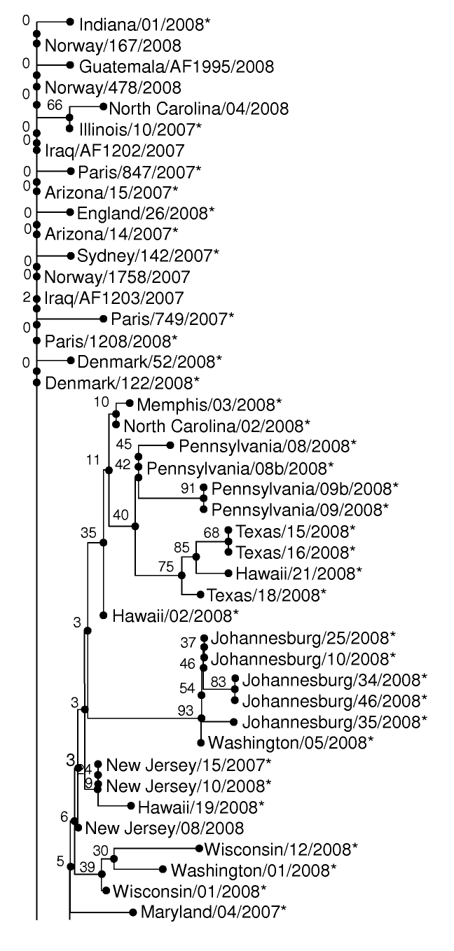

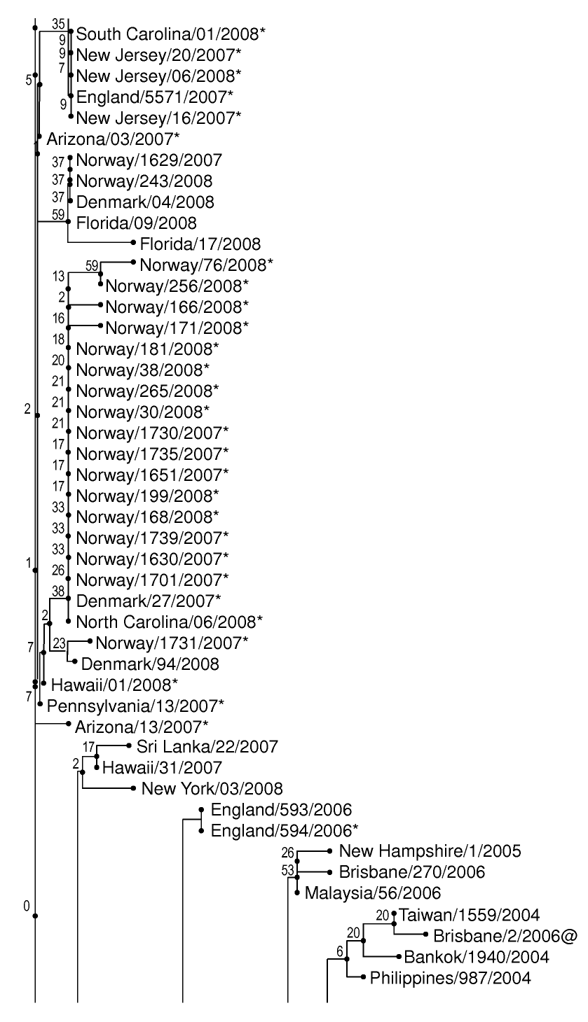

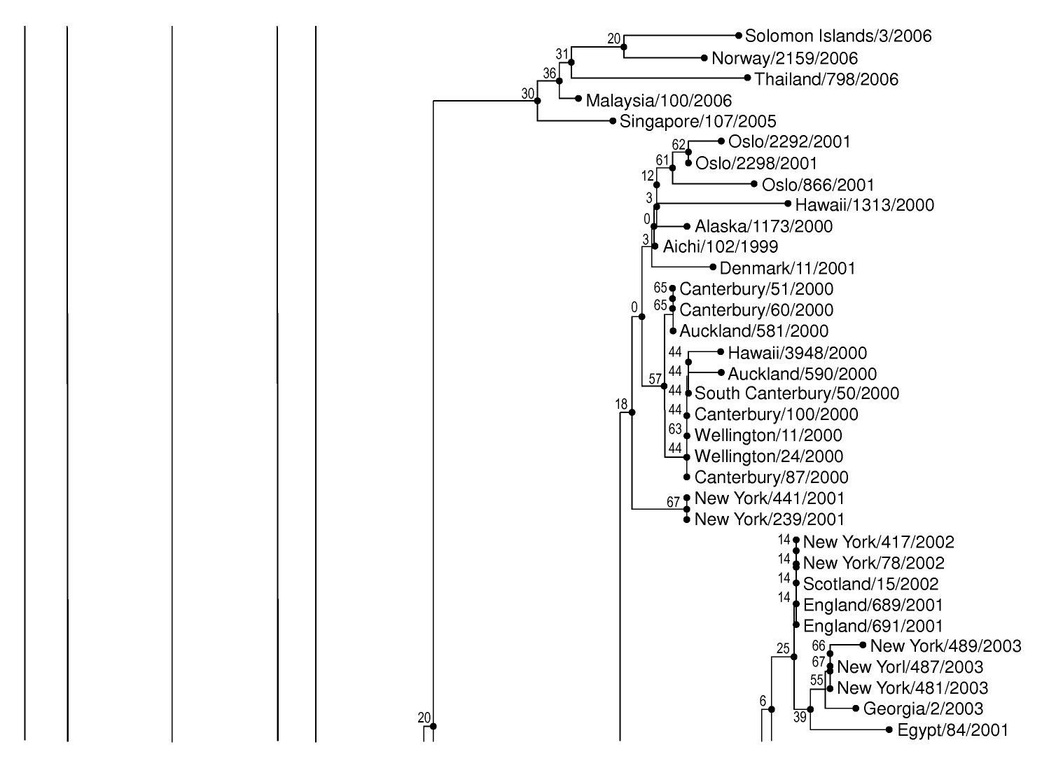

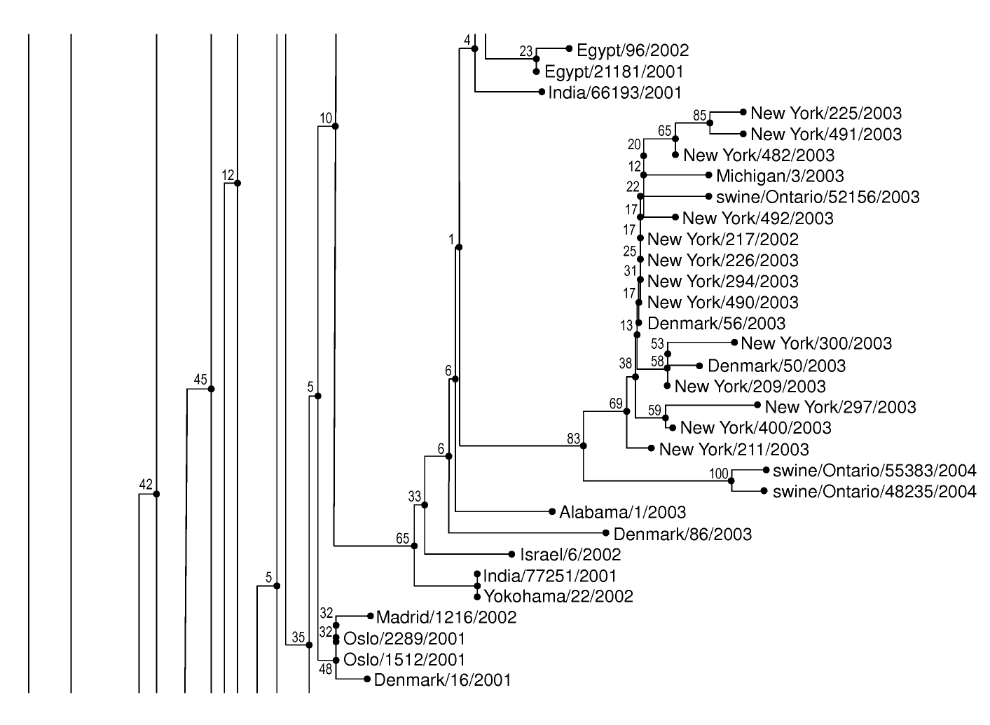

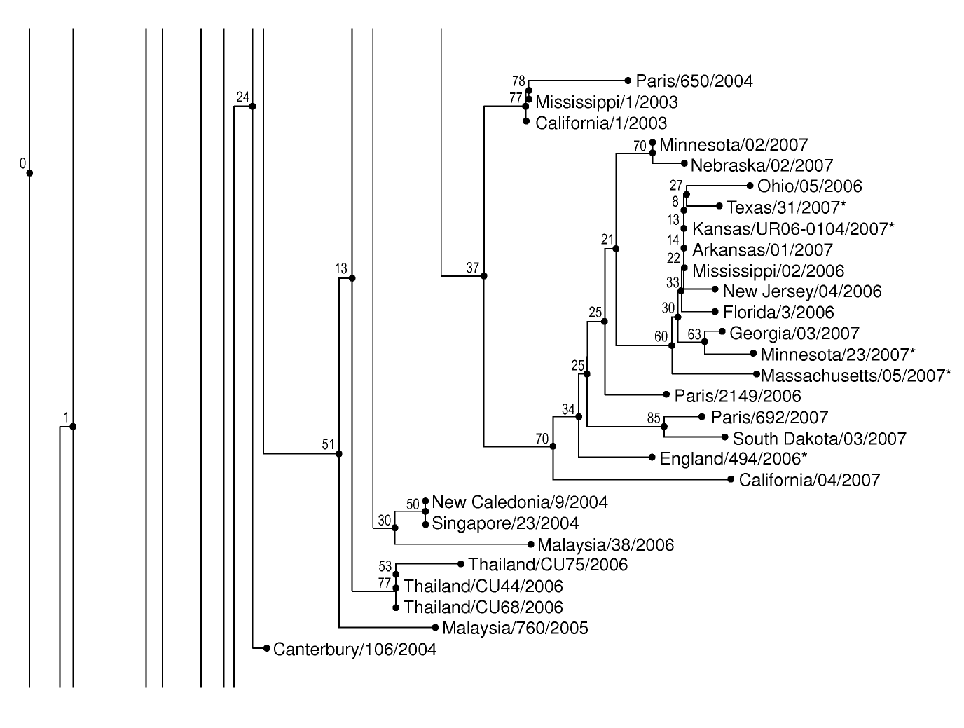

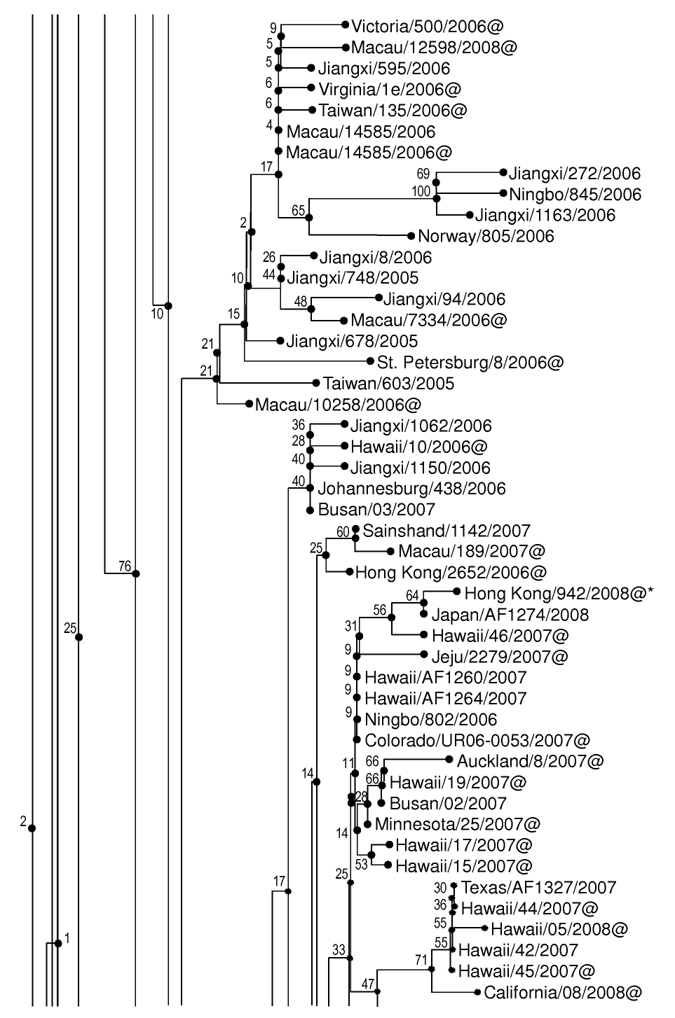

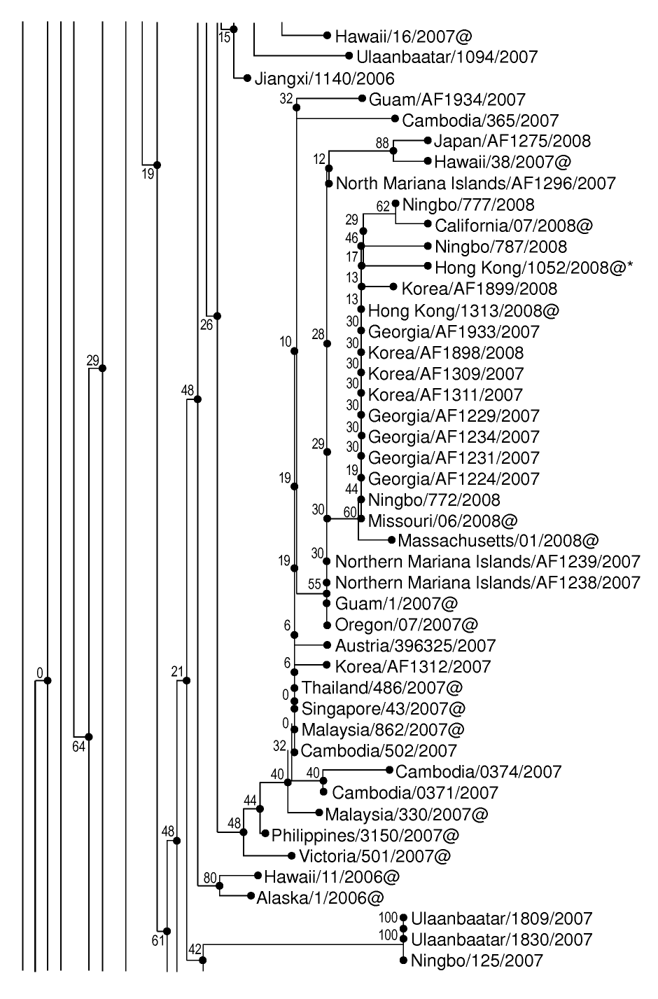

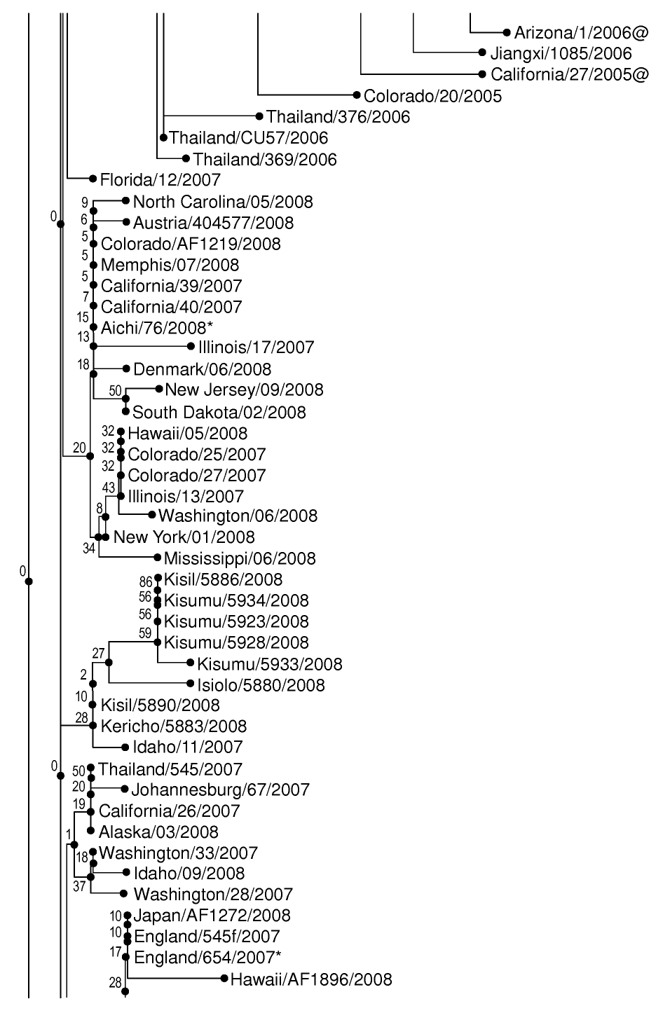

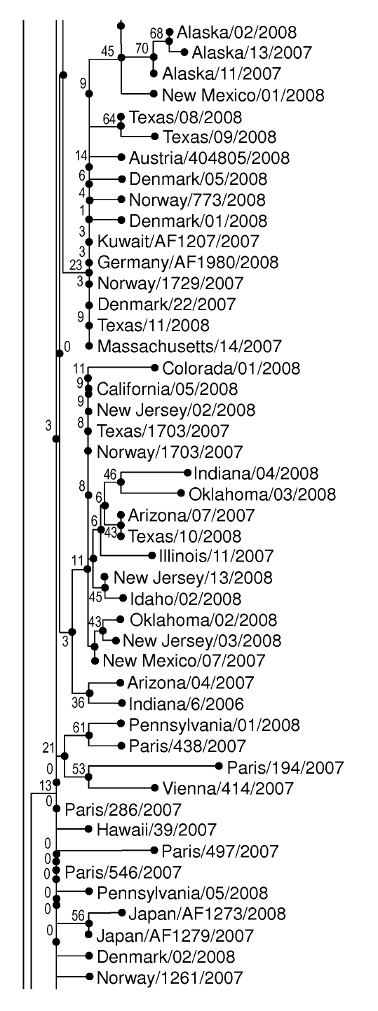

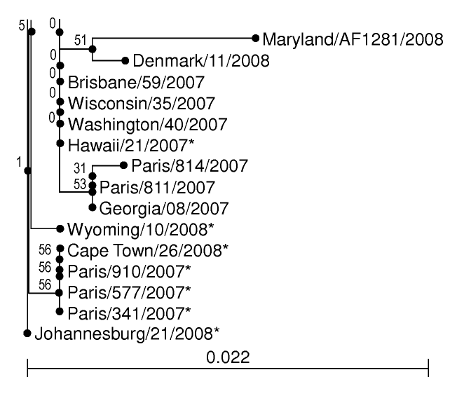

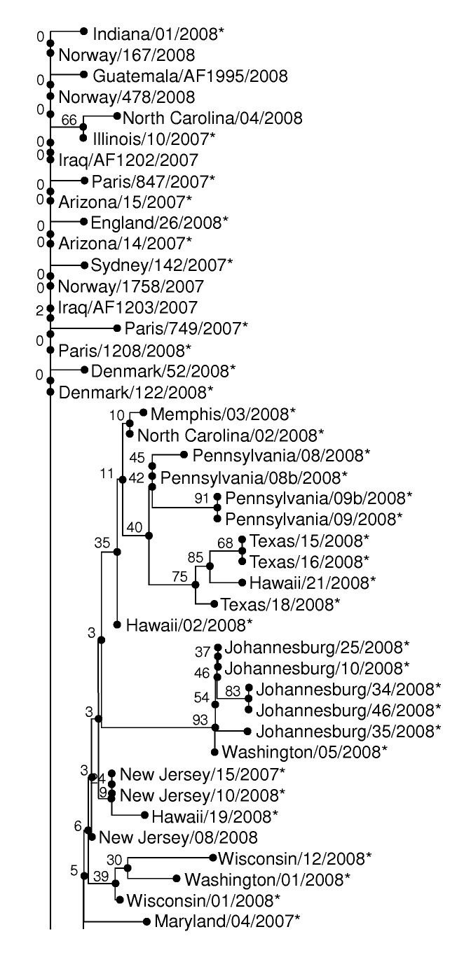

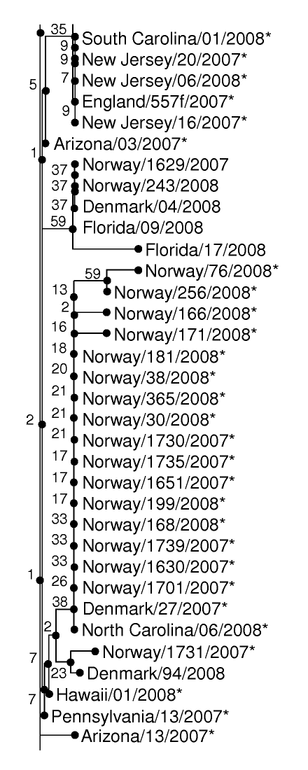

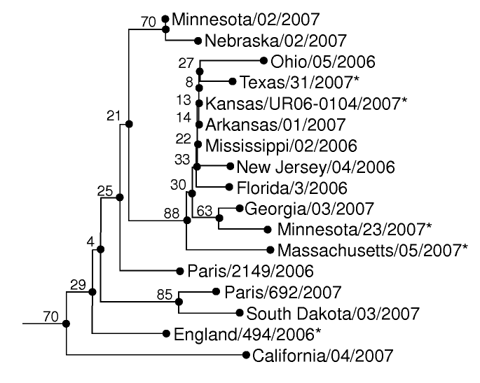

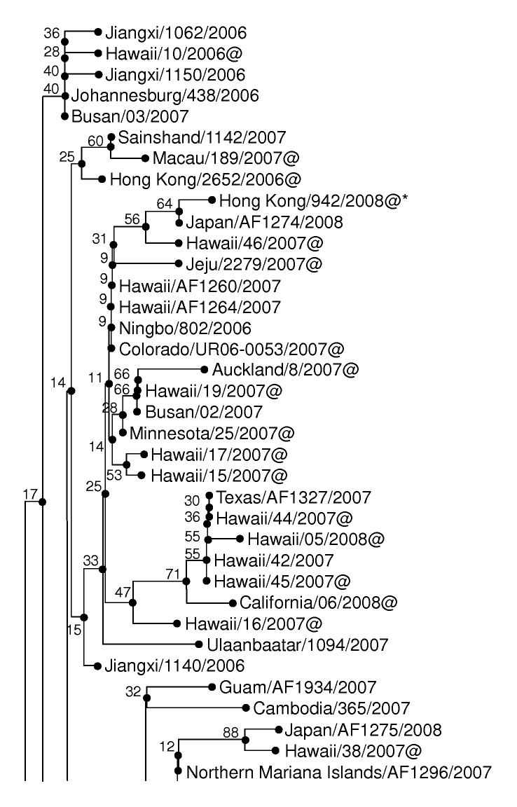

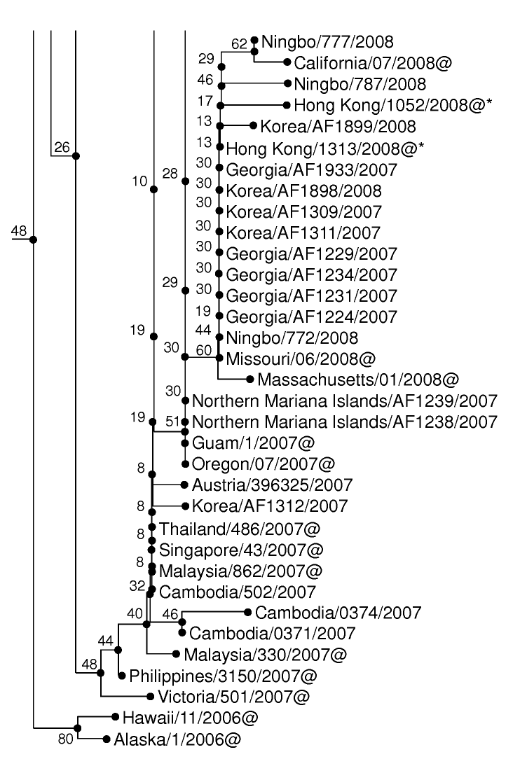

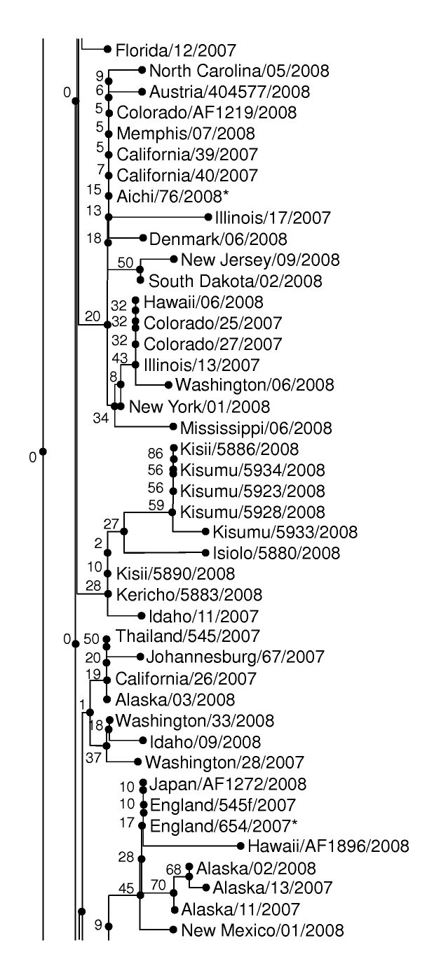

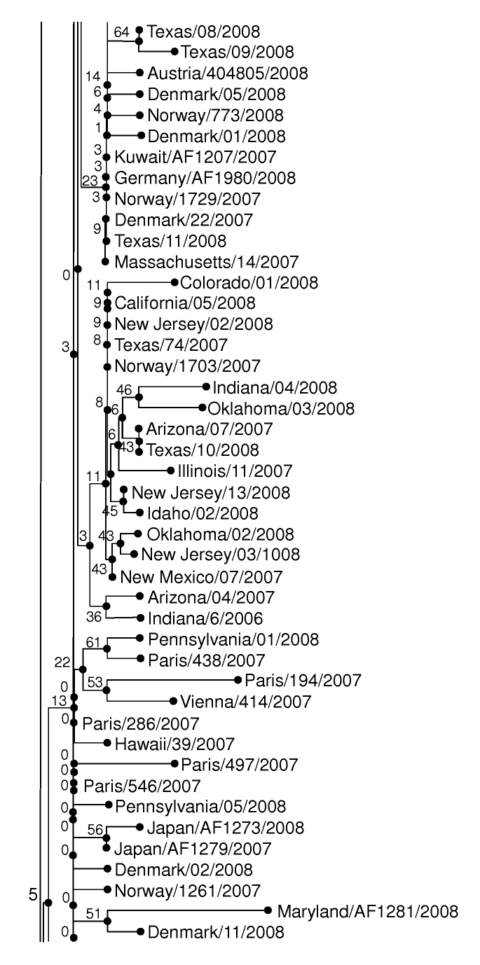

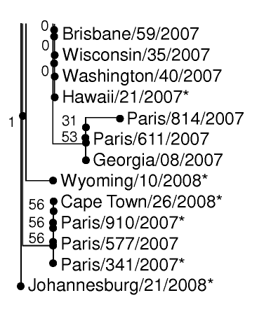

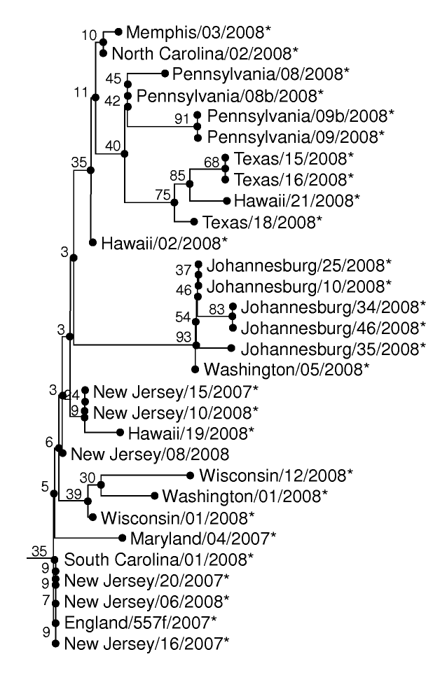

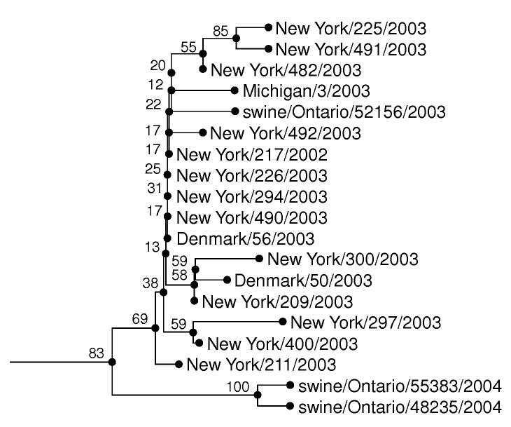

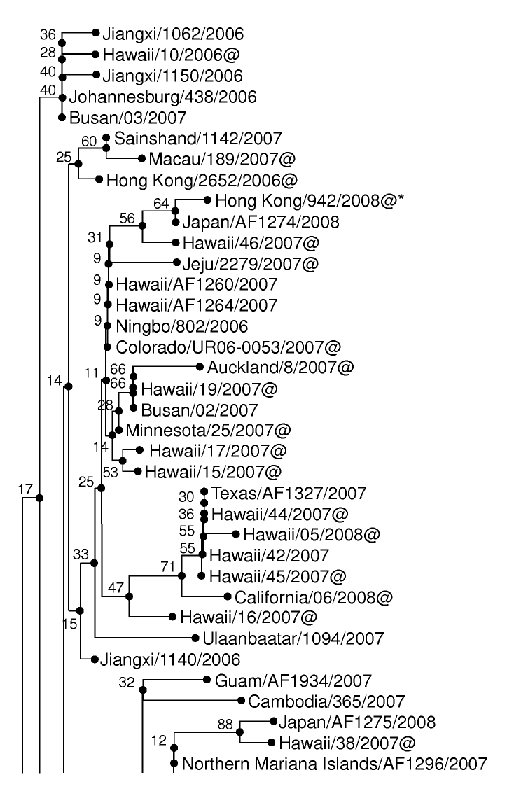

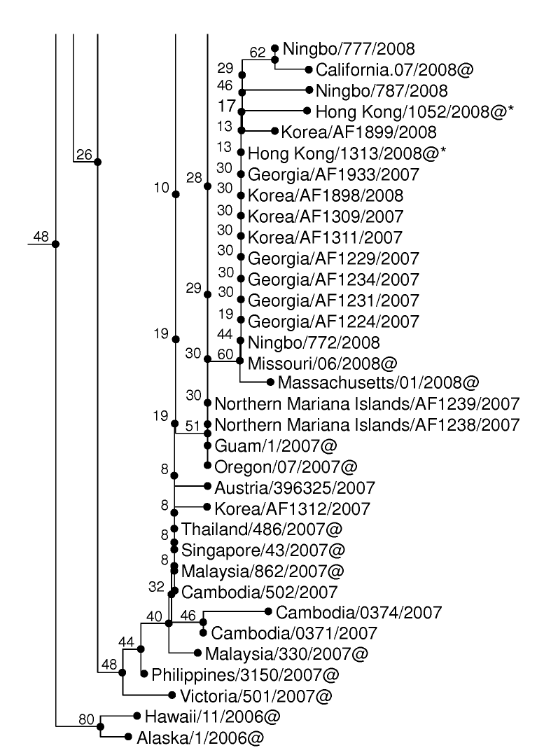

FIG. 2B details some of the NA changes associated with this emergence. Two tandem polymorphisms, D344N and D354G (encoded by G1030A and A1061G, respectively) defined Clade 2B* that was widespread by the end of 2007, and in early 2008 led to frequencies greater than 50% of H274Y in H1N1, which were reported in Norway (see, e.g., Hauge S H, et al., Emerg Infect Dis. 2009 February. DOI: 10.3201/eid1502.081031, the entire contents of which are expressly incorporated herein by reference). These two polymorphisms were in clade 1 H1N1 isolates from 2001/2002. Levels of H1N1 declined after the 2003/2004 season, and began to emerge in Asia in 2005/2006. Clade 2C emerged in 2006/2007 and had acquired D344N and D354G, as well as a synonymous polymorphism, G1041A. Clade 1 emerged worldwide in the 2006/2007 season, but did not have the three polymorphisms. Clade 2B expanded in 2007/2008 and the dominate sub-clade, including Brisbane/59 had D344N. However, Clade 2B* emerged with the same three polymorphisms which appeared in the prior season in Clade 2C. Moreover, flanking regions created a 80 BP stretch of identity between Clade 2B and Clade 2C, supporting acquisition by homologous recombination.

Phylogenetic analysis of HA is presented in FIG. 2. FIG. 2A supports the data in the NA phylogram, showing multiple independent introductions in clade 2C, clade 1, and early clade 2B, followed by the emergence of Clade 2B*. Clade 2C had three receptor binding domain changes, R192M, A193T, and T197K (FIG. 2B). A sub-clade of Clade 2B*, Clade 2B** emerged in multiple locations in the United States as well as England. It had acquired A193T

The high levels reported in several countries in Europe were surpassed in the 2008 influenza season in the southern hemisphere. Resistance levels rose to 100% in South Africa (see, e.g., Besselaar T G, et al., Emerg Infect Dis: 14,1809-1810 (2008), the entire contents of which are expressly incorporated herein by reference), suggesting additional changes were creating strong selection pressure. HA sequences from South Africa identified a dominant sub-clade with five polymorphisms clustered around receptor binding domain position 190 (H3 numbering). Four of these nucleotide changes produce three non-synonymous polymorphisms, N187D, G189N, and A193T (see FIG. 2B). All three changes were present in other recent H1N1 isolates. A193T was present in Clade 2B* in the 2007-2008 season. N187D was in an H1N1 clade 2C isolate from Hong Kong. G189N was encoded by two adjacent nucleotide changes that were in clade 2B isolates from Kenya.

The presence of A193T in a sub-set of isolates in Clade 2B* and the dominant sub-clade in South Africa, was extended to isolates from the current season. The fixing of H274Y in clade 2B in South Africa was extended to North America and Europe this season. All clade 2B isolates reported to date in the United States and Canada have H274Y. As seen in FIG. 2, all isolates from the United States this season are on the same branch and all isolates have A193T, suggesting this change drove the fixing of H274Y in H1N1 clade 2B. The isolates have additional changes at position 190 (D190N), as well as the adjacent position (G189V), which may be linked to further selection. A recent report from Japan identified sequences which matched isolates from the United States. One group had G189V, A193T, and H186R. Another series had G189N and A193T, while another series had G189A and A193T, supporting the importance of A193T.

The fixing and spread of H274Y in clade 2B, is similar to the emergence of adamantine resistance associated with S31N in MP2 in H1N1 clade 2C. Although S31N was becoming fixed in a sub-clade in China in the 2006/2007 season, its spread to multiple countries, including the United States was linked to a 2C sub-clade that had acquired a number of changes near position 190 (R192M, A193T, and T197K). This sub-clade represented approximately 10% of H1N1 isolates in the United States, and all had S31N. Moreover, as seen in FIG. 2, the same sub-clade was found in Air Force personal or dependents in South Korea, Japan, Marian Islands, Hawaii, and Georgia supporting a significant expansion of this sub-clade.

The presence of HA A193T in clade 2B isolates that had fixed NA H274Y as well as clade 2C isolates that had fixed M2 S31N suggests that the spread of the resistance was due to genetic hitch hiking of the resistance markers with HA receptor binding domain changes. This mechanism is supported further by the fixing of M2 S31N in H3N2, which was associated with two HA receptor binding domain changes, D225N and S193F. The association of position 193 with the fixing and spread of antiviral resistance strongly supports genetic hitch hiking as the mechanism of the fixing of these three examples of antiviral resistance. Moreover, recent Clade 2C isolates from Hong Kong have both H274Y on NA and S31N on M2.

The acquisition of changes in the H1N1 is most easily explained by homologous recombination. The changes in the receptor binding domain were appended onto a clade 2B genetic background that had A193T. One of the changes, N187D, is rare but is found in a 2008 clade 2C isolate in Hong Kong. The other change is also rare and requires tandem nucleotide changes. The same two changes are found in 2008 H1N1 isolates from Kenya. Acquisition of these changes by independent copy errors is unlikely since the donor sequences are in Africa in 2008, and the changes were among a limited number of changes on the South African sub-clade.

Acquisition by homologous recombination is also supported by the dominant changes in NA and HA. The tandem changes of D344N and D354G are present on three different genetic backgrounds, H1N1 in circulation between 2001 and 2003, clade 2C emergence with S31N in 2007, and clade 2B emergence with H274Y in 2007 (see, e.g., Rameix-Welti et al., Enzymatic properties of the neuraminidase of seasonal H1N1 influenza viruses provide insights for the emergence of natural resistance to oseltamivir. PLoS Pathog:4:e1000103 (2008), the entire contents of which are expressly incorporated herein by reference). Similarly the emergence of these two recent sub-clades was associated with the acquisition of A193T on HA, which was also in H1N2 isolates that emerged in 2003.

The exchanges of genetic information between clade 2B and clade 2C are facilitated by co-circulation. In addition to the presence of N187D on clade 2B in South Africa and clade 2C in Hong Kong, one of the Hong Kong isolates is a reassortant, with a clade 2C HA and a clade 2B NA. Moreover, this isolate, and others collected over the summer in Hong Kong have a clade 2C HA and M2 which has H274Y in NA (both clade 2B and clade C) as well as S31N on M2, signaling additional exchanges of polymorphisms leading to the emergence of H1N1 which is resistant to oseltamivir and the adamantanes.

Moreover, H274Y was associated with independent introductions onto multiple independent H1N1 backgrounds (clade 2C, clade 1, and clade 2B) which are also most easily explained by homologous recombination. The introduction onto multiple backgrounds followed by widespread fixing is similar to a synonymous change on H1N1 clade 2.2 NA, G743A (see, e.g., Niman, H L, et al. Concurrent acquisition of a single nucleotide polymorphism in diverse influenza H5H1 clade 2.2 sub-clades. Available from Nature Precedings <http://hdl:hdl.handle.net/10101/npre.2008.459.4> (2008), the entire contents of which are incorporated herein by reference). This polymorphism was on a geographically and genetically restricted sub-clade in 2006. However, in early 2007 it appeared on multiple clade 2.2 genetic backgrounds in Russia, Egypt, Kuwait, Ghana, and Nigeria. The change was then fixed on clade 2,2,3 which evolved from its introduction in Kuwait in early 2007 and became fixed in Europe in the 2007/2008 season.

The movement of single nucleotide polymorphisms via recombination between closely related sequences is not unexpected. Earlier analysis of swine influenza identified multiple examples of recombination involving long stretches of identity. However, multiple recombination events led to shorter regions from a given parental sequence (see, e.g., Niman, H L, Swine influenza A evolution via recombination—genetic drift reservoir. Available from Nature Precedings <http://hdl.handle.net/10101/npre.2007.385.1> (2007); Hea et al., Virology 380,12-20 (2008); and Krasnitz et al., J Virol 82,8947-8950 (2008), the entire contents of each of which are expressly incorporated herein by reference). Shorter regions were identified in an analysis of human influenza (see, e.g., Boni, M F, et al., J Virol 82,4807-4811(2008), the entire contents of which are expressly incorporated herein by reference). Similarly, the exchanges have also been seen for deletions. A three BP deletion appended onto H5H1 clade 2.2 in Egypt was identical to the deletion appended onto a clade 7 background in China (see, e.g., Niman H L, et al. H5H1 Clade 2.2 Polymorphism tracing identifies influenza recombination and potential vaccine targets in Options for the Control of Influenza VI, J M Katz, ed, International Medical Press, London, pp 436-438 (2008), the entire contents of which are expressly incorporated herein by reference). Moreover, recombination between closely related sequences, such as H1N1 sub-clades as described in this report, or between sub-clades of H5H1 described above, produces exchanges of single nucleotide polymorphisms.

The fixing of antiviral resistance follows a general mechanism for rapid viral evolution. Recombination places a given polymorphism onto multiple genetic backgrounds. Most of the initial introductions failed to become dominant. This was seen in the United States in the first clade 2B isolates which were in Hawaii. These isolates formed a branch with other isolates from Hawaii and California, but this sub-clade did not spread beyond the initial two isolates. The pattern was repeated for a Florida isolate that formed a separate branch, but the H274Y on this background also did not spread. Similar results were seen for an isolate from France, which spread to a minor sub-clade in South Africa. The sub-clade in Norway also did not spread beyond adjacent countries (Finland and Denmark). However, the acquisition of H274Y by isolates with A193T in 2007/2008 was followed by fixing in Europe and North America in the following season, supporting the emergence and spread by genetic hitch hiking.

This mechanism has significant theoretical and practical considerations. The acquisition of polymorphisms that have a circulated previously allows for predictions based on the present and past frequency of the polymorphisms as well as likely interactions between various genomes. In the fixing of H274Y, the polymorphism appeared on multiple H1N1 sub-clades before it was fixed throughout the northern hemisphere. The fixing was associated with the acquisition of a number of additional polymorphisms on NA and HA. The acquisition of A193T was preceded by its association with the fixing of S31N in M2 of H1N1 clade 2C (see, e.g., Barr, I G, et al., Antiviral Res 75,173-176 (2007), the entire contents of which are expressly incorporated herein by reference), and a change at the same position on H3, S193F, was associated with the fixing of S31N on H3N2 (see, e.g., Medeiros, R et al., Mol. Biol. Evol 24,1811-1820 (2007) and Barr, I G et al., Antiviral Res 73,112-117 (2007), the entire contents of each of which are expressly incorporated herein by reference). Thus, the appearance of A193T at the end of 2007 was a signal that it would play a role in the spread of H274Y in the following season.

The role of A193T in the emergence of H274Y may extend beyond an immunological escape mechanism. The position has been reported to be associated with a change in tissue tropism, which affects affinity and is expressed in a species specific manner (see, e.g., Medeiros, et al., Archives of Virol:149,1663-1671 (2004), the entire contents of which are expressly incorporated herein by reference). Moreover, the dominant H1N1 reported to date for this season includes an adjacent change, H196R, which also has been linked to an affinity changes in H5H1 on a clade 1 background in Vietnam, as well as a clade 2.2 background in Iraq. In both instances the change was Q196R (see, e.g., Yamada, S, et al., Nature 444, 378-382 (2006), the entire contents of which are expressly incorporated herein by reference). The H5H1 studies also associated position 186 in changes in affinity for receptors on target cells, and position 190 has been associated with species specific changes (see, e.g., Stevens, J, et al., J Mol Biol 355, 1143-1155 (2006), the entire contents of which are expressly incorporated herein by reference).

Therefore, changes in or near the receptor binding domain in HA are candidates for vaccine targets. A193T was circulating in late 2007 and early 2008 in the United States and England, signaling its fixing in the following season in the northern hemisphere. Although the selection of Brisbane/59 for the vaccine target was considered a “match” for the H1N1 in the 2008/2009 season, it did not contain A193T. Moreover, A193T has not been included in any of the H1N1 vaccine targets, raising the possibility that its absence in recent vaccines played a role in its emergence in clade 2C in Asia, followed by clade 2B worldwide. Similarly, one of the NA changes associated with the emergence of H274Y, D354G, as well as H274Y itself have not been present in recent H1N1 vaccine targets.

In addition to the prediction of vaccine targets, the pattern of polymorphism acquisition can be used to predict the fixing of drug resistance, such as H274Y for oseltamivir, or S31N for adamantanes in H1N1 or H3N2. The emergence of the resistance has led to a more robust influenza database, most notably for HA, NA, and MP gene segments. However, an expanded database with sequences from more locations in Asia and Africa should create more accurate predictions of emerging polymorphisms for use in predicting vaccine targets and fixing of antiviral resistance.

### REFERENCES

1. World Health Organization Collaborating Centers. Influenza A(H1N1) virus resistance to oseltamivir—2008/2009 influenza season, northern hemisphere. December 2008.
2. Centers for Disease Control and Prevention—Korea. Antiviral resistant influenza A viruses isolated in Korea during 2008-2009 season—Oseltamivir resistance to A/H1N1 in Korea. January 2009.
3. Lackenby, A, et al. Emergence of resistance to oseltamivir among influenza A(H1N1) viruses in Europe. Eurosurveillance, 13, January 2008.
4. de Jong M D, et al. Oseltamivir resistance during treatment of influenza A (H5H1) infection. N Engl J Med 353,2667-2672 (2005).
5. Kiso, M K, et al. Resistant influenza A viruses in children treated with oseltamivir: descriptive study. Lancet: 364,759-765 (2004).
6. Ives, J A, et al. The H274Y mutation in the influenza A/H1N1 neuraminidase active site following oseltamivir phosphate treatment leave virus severely compromised both in vitro and in vivo. Antivir. Res:55,307-317 (2002).
7. Herlocher M L et al. Influenza viruses resistant to the antiviral drug oseltamivir: transmission studies in ferrets. J Infect Dis:190,1627-1630 (2004).
8. Sheu T G, et al. Surveillance for neuraminidase inhibitor resistance among human influenza A and B viruses circulating worldwide from 2004 to 2008. Antimicrob Agents Chemother;52,3284-3292 (2008).
9. Hauge S H, S Dudman, K Borgen, A Lackenby, & O Hungnes. Oseltamivir-resistant influenza viruses A (H1N1), Norway, 2007-08. Emerg Infect Dis. 2009 February. DOI: 10.3201/eid1502.081031.
10. Besselaar T G, et al. Widespread oseltamivir resistance in influenza A viruses (H1N1), South Africa. Emerg Infect Dis:14,1809-1810 (2008).
11. Rameix-Welti M A, V Enouf, F Cuvelier, P Jeannin, & S van der Werf. Enzymatic properties of the neuraminidase of seasonal H1N1 influenza viruses provide insights for the emergence of natural resistance to oseltamivir. PLoS Pathog:4:e1000103 (2008).
12. Niman, H L, et al. Concurrent acquisition of a single nucleotide polymorphism in diverse influenza H5H1 clade 2.2 sub-clades. Available from Nature Precedings <http://hdl:hdl.handle.net/10101/npre.2008.459.4> (2008)
13. Niman, H L. Swine influenza A evolution via recombination—genetic drift reservoir. Available from Nature Precedings <http://hdl.handle.net/10101/npre.2007.385.1> (2007).
14. Hea, C-Q, et al. Homologous recombination evidence in human and swine influenza A viruses. Virology 380,12-20 (2008).
15. Krasnitz, M, A J Levine & R Rabadan. Anomalies in the influenza virus genome database: new biology or laboratory errors? J Virol 82,8947-8950 (2008).
16. Boni, M F, Y Zhou, J K Taubenberger & E C Holmes. Homologous recombination is very rare or absent in human influenza A virus. J Virol 82,4807-4811 (2008).
17. Niman H L, et al. H5H1 Clade 2.2 Polymorphism tracing identifies influenza recombination and potential vaccine targets in Options for the Control of Influenza VI, J M Katz, ed, International Medical Press, London, pp 436-438 (2008).
18. Barr, I G, A C Hurt, N. Deeda, P. Iannello, C. Tomasov, & N. Komadina. The emergence of adamantane resistance in influenza A(H1) viruses in Australia and regionally in 2006. Antiviral Res 75,173-176 (2007).
19. Medeiros, R et al. The Genesis and Spread of Reassortment Human Influenza A/H3N2 Viruses Conferring Adamantane Resistance. Mol. Biol. Evol 24,1811-1820 (2007).
20. Barr, I G et al. Increased adamantane resistance in influenza A(H3) viruses in Australia and neighbouring countries in 2005. Antiviral Res 73,112-117 (2007).
21. Medeiros, R, N Naffakh, J C Manuguerra, & S. van der Werf. Binding of the hemagglutinin from human or equine influenza H3 viruses to the receptor is altered by substitutions at residue 193. Archives of Virol: 149,1663-1671 (2004).
22. Yamada, S, et al. Haemagglutinin mutations responsible for the binding of H5H1 influenza A viruses to human-type receptors. Nature 444, 378-382 (2006).
23. Stevens, J, et al. Glycan microarray analysis of the hemagglutinins from modern and pandemic influenza viruses reveals different receptor specificities. J Mol Biol 355, 1143-1155 (2006).

The contents of all references, patents and published patent applications cited throughout this application are hereby incorporated herein by reference in their entirety.

### Equivalents

Those skilled in the art will recognize, or be able to ascertain using no more than routine experimentation, many equivalents to the specific embodiments of the invention described herein. Such equivalents are intended to be encompassed by the following claims.

1. A method of predicting emergence or expansion of a drug resistant viral strain sequence from sequences of a first parental viral strain and a second parental viral strain, comprising:
identifying a first parental viral strain sequence comprising one or more sequences correlated with a characteristic of the virus;
identifying a second parental viral strain sequence lacking one or more of the one or more sequences of the first parental viral strain; and
predicting drug resistant viral strain sequences capable of arising from a genetic transfer event comprising replacement of a second parental viral strain sequence with a first parental viral strain sequence,
such that emergence or expansion of a drug resistant viral strain sequence having a characteristic of the parental viral strain is predicted.

2. The method of claim 1, wherein the viral strains are influenza viruses.

3. The method of claim 1, wherein the characteristic is genotypic, phenotypic, molecular, epidemiological, clinical, or pathological.

4. The method of claim 3, wherein the molecular characteristic is a nucleic acid alteration or amino acid alteration.

5. The method of claim 4, wherein the nucleic acid or amino acid alteration is in an influenza sequence selected from the group consisting of HA, NP, NA, PA, PB1, PB2, M1, M2, NS1, and NS2, or combinations thereof.

6-20. (canceled)

21. The method of claim 4, wherein the amino acid alteration is an alteration in amino acid residue 196.

22. The method of claim 21, wherein said alteration in amino acid residue 196 is Histidine to Arginine or Glutamine to Arginine.

23. The method of claim 4, wherein the nucleic acid or amino acid alteration is in an influenza M2 sequence.

24. The method of claim 23, wherein the alteration is an amino acid residue in an influenza M2 sequence.

25. The method of claim 24, wherein said alteration is at amino acid residue 31.

26. The method of claim 25, wherein said alteration of amino acid 31 is Serine to Asparagine.

27. The method of claim 26, further comprising an additional alteration at amino acid residue in an influenza HA sequence selected from the group consisting of: amino acid residue 192, amino acid residue 193, and amino acid residue 197.

28-77. (canceled)

78. A composition comprising a nucleic acid or polypeptide sequence, wherein the nucleic acid or polypeptide sequence is selected from the group consisting of: an altered influenza NA sequence, an altered influenza M2 sequence, and an altered influenza HA sequence.

79. The composition of claim 78, wherein the alteration is an amino acid residue in an influenza NA sequence.

80. The composition of claim 79, wherein said alteration is at amino acid residue 274.

81. The composition of claim 80, wherein said alteration of amino acid 274 is histidine to tyrosine.

82. The composition of claim 79, wherein the alteration is at amino acid residue 294.

83. The composition of claim 82, wherein said alteration of amino acid 294 is asparagine to serine.

84-87. (canceled)

88. The composition of claim 78, wherein the alteration is an amino acid residue in an influenza HA sequence.

89. The composition of claim 88, wherein said alteration is at amino acid residue 193.

90. The composition of claim 80, wherein said alteration of amino acid 193 is alanine to threonine.

91. The composition of claim 78, wherein the alteration is an amino acid residue in an influenza M2 sequence.

92. The composition of claim 91, wherein said alteration is at amino acid residue 31.

93. The composition of claim 92, wherein said alteration of amino acid residue 31 is Serine to Asparagine.

94-95. (canceled)

96. A method of immunizing an animal or human subject against influenza comprising administering to the subject the composition of claim 78.

97-111. (canceled)
